(19) United States
(12) Patent Application Publication
(10) Pub. No.: US 2008/0057086 A1
(43) Pub. Date: Mar. 6, 2008

(54) COLON-TARGETED ORAL FORMULATIONS OF CYTIDINE ANALOGS

(75) Inventor: Jeffrey B. Etter, Boulder, CO (US)

Correspondence Address: SWANSON & BRATSCHUN, L.L.C., 8210 SOUTHPARK TERRACE, LITTLETON, CO, 80120, US

(73) Assignee: PHARMION CORPORATION, Boulder, CO (US)

(21) Appl. No.: 11/849,958

(22) Filed: Sep. 4, 2007

Related U.S. Application Data
(60) Provisional application No. 60/824,320, filed on Sep. 1, 2006.

(51) Int. Cl. A61K 9/00; A61K 31/7068; A61P 43/00

(52) U.S. Cl. 424/400; 514/49

(57) ABSTRACT

The present invention provides an oral formulation of a cytidine analog, including, 5-azacytidine, for delivery to the lower gastrointestinal tract, including, the large intestine; methods to treat diseases associated with abnormal cell proliferation by treatment with the oral formulations of the present invention; and methods to increase the bioavailability of a cytidine analog upon administration to a patient by providing an oral formulation of the present invention.

### RELATED APPLICATIONS

This application is a non-provisional of U.S. Patent Application Ser. No. 60/824,320, filed Sep. 1, 2006, entitled “Oral Formulations of Cytidine Analogs”, which is incorporated by reference herein in its entirety.

### BACKGROUND OF THE INVENTION

Cellular proliferative disorders are responsible for numerous diseases resulting in major morbidity and mortality and have been intensively investigated for decades. Cancer now is the second leading cause of death in the United States, and over 500,000 people die annually from this proliferative disorder.

Nucleoside analogs have been used clinically for the treatment of viral infections and proliferative disorders for decades. Most of the nucleoside analog drugs are classified as antimetabolites. After they enter cells, nucleoside analogs are successively phosphorylated to nucleoside 5′-monophosphates, 5′-diphosphates, and 5′-triphosphates. In most cases, nucleoside triphosphates are the chemical entities that inhibit DNA or RNA synthesis, either through a competitive inhibition of polymerases or through incorporation of modified nucleotides into DNA or RNA sequences. Nucleosides may act also as their diphosphates.

5-Azacytidine (also known as azacitidine and 4-amino-1-β-D-ribofuranosyl-1,3,5-triazin-2(1H)-one; Nation Service Center designation NSC-102816; CAS Registry Number 320-67-2) has undergone NCl-sponsored trials for the treatment of myelodysplastic syndromes (MDS). See Kornblith et al., J. Clin. Oncol. 20(10): 2441-2452 (2002) and Silverman et al., J. Clin. Oncol. 20(10): 2429-2440 (2002). 5-Azacytidine may be defined as having a molecular formula of C₈H₁₂N₄O₅, a relative molecular weight of 244.21 and a structure of:

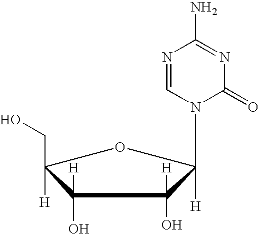

Azacitidine (also referred to herein as 5-azacytidine herein) is a nucleoside analog, more specifically a cytidine analog. 5-azacytidine is an antagonist of its related natural nucleoside, cytidine. 5-azacytidine, as well as decitabine, i.e., 5-aza-2′-deoxycytidine, are antagonists of decitabine's related natural nucleoside, deoxycytidine. The only structural difference between the analogs and their related natural nucleosides is the presence of nitrogen at position 5 of the cytosine ring in place of oxygen.

Other members of the class of deoxycytidine and cytidine analogs include arabinosylcytosine (Cytarabine), 2′-deoxy-2′,2′-difluorocytidine (Gemcitabine), 5-aza-2′-deoxycytidine (Decitabine), 2(1H) pyrimidine riboside (Zebularine), 2′,3′-dideoxy-5-fluoro-3′thiacytidine (Emtriva), N⁴-pentyloxycarbonyl-5′-deoxy-5-fluorocytidine (Capecitabine), 2′-cyclocytidine, arabinofuanosyl-5-azacytidine, dihydro-5-azacytidine, N⁴-octadecyl-cytarabine, elaidic acid cytarabine, and cytosine 1-β-D-arabinofuranoside (ara-C).

In general, oral delivery of members of this class of compounds has proven difficult due to combinations of chemical instability, enzymatic instability, and/or poor tissue permeability. For example, these compounds are known to be acid labile and thus unstable in the acidic gastric environment. In the case of 5-azacytidine, ara-C, decitabine and gemcitabine, an enzyme thought to be responsible for a significant portion of drug metabolism is cytidine deaminase. Strategies to improve the oral bioavailability of this drug class have included the use of prodrugs to modify chemical and enzymatic instability, and/or the use of enzymatic inhibitors.

For example, DeSimone et al describe the ability of 5-azacytidine to induce fetal hemoglobin production in baboons when administered via the intravenous (IV), subcutaneous (SC), or perioral (PO) route. In the case of PO administration the author states that co-administration of THU (tetrahydrouridine) was necessary to achieve fetal hemoglobin induction, however no specific data is provided on the doses or responses observed without THU. 5-azacytidine doses ranged from 0.25 mg/kg/d to 8 mg/kg/d with co-administration of 20 mg/kg/d THU. Administration of THU alone was shown to result in a significant decrease in peripheral cytidine deaminase activity.

Neil, et al describe the effect of THU on the pharmacokinetics and pharmacodynamics of inter peritoneal (I.P.) and peri oral (P.O.) 5-azacytidine when administered to leukemic mice. Pharmacokinetic parameters were determined using a bioassay that did not discriminate between 5-azacytidine and its degradation and metabolism products. Inclusion of THU with IP administration had little effect on the clearance or degradation of 5-azacytidine. Inclusion of THU with PO administration significantly increased both Cₘₐₓ and t_{1/2}. In both acute and chronic IP dosing the inclusion of THU did not influence the pharmacodymamic effects of 5-azacytidine except at the highest chronic dose which was toxic. Conversely, co-administration of THU with PO 5-azacytidine resulted in increased efficacy at all doses except the highest chronic dose which was again toxic.

Dunbar, et al describe the administration of 5-azacytidine via IV and PO routes for increased production of total hemoglobin in a β⁰-thalassemic patient. Doses of 2 mg/kg/d IV resulted in a measurable increase to hemoglobin levels. Administration of 2 mg/d tid (three times daily) PO with co-administration of THU did not result in increased hemoglobin levels.

Dover, et al describe administration of 5-azacytidine via the SC and PO routes for increased production of total hemoglobin, fetal hemoglobin and F cells in sickle cell patients. 5-azacytidine oral bioavailability was assessed by clinical response only. Dover reports that oral doses of 5-azacytidine (2 mg/kg/d) alone or THU (200 mg/d) alone did not result in increased F reticulocyte production. However oral doses of 200 mg/d of THU were observed to result in a significant suppression of peripheral cytidine deaminase activity for several days post administration. When 5-azacytidine was co-administered with THU good clinical response was observed as determined by total hemoglobin, fetal hemoglobin and F cell levels. In fact comparable clinical response was observed with doses of 2 mg/kg/d SC without THU versus 0.2 mg/kg/d PO with co-administration of 200 mg/d THU. Oral doses of 5-azacytidine and THU were prepared by encapsulation at the clinical site. No information was provided with respect to excipients.

Efforts to increase bioavailability of this class of compounds have also been described in, for example, U.S. Patent Application Publication No. 2004/0162263 (Sands, et al.) In this publication, delivery of 5-azacytidine in an enteric-coated formulation are disclosed such that the drugs are preferably absorbed in the upper regions of the small intestine, such as the jejunum. All U.S. patents and patent publications referenced herein are incorporated by reference herein in their entireties.

Despite these efforts, a need remains for more effective methods and compositions which increase oral bioavailability of this class of compounds.

### BRIEF DESCRIPTION OF THE DRAWINGS

FIG. 1 represents a graph showing Absolute Mucosal to Serosal Permeability of 5-azacytidine in Human Intestinal Tissue with and without Enzymatic Inhibition.

FIG. 2 represents a graph showing Relative Mucosal to Serosal Permeability of 5-azacytidine in Human Intestinal Tissue with and without Enzymatic Inhibition with Respect to Atenolol.

FIG. 3 represents a graph showing Absolute Mucosal to Serosal Permeability of 5-azacytidine in Human Colonic Tissue with Various Concentrations of TPGS or Labrafil without Enzymatic Inhibition.

FIG. 4 represents a graph showing Relative Mucosal to Serosal Permeability of 5-azacytidine in Human Colonic Tissue with Various Concentrations of TPGS or Labrafil without Enzymatic Inhibition.

FIG. 5 shows concentration vs time profiles of individual subjects administered an oral formulation of the present invention.

FIG. 6 shows concentration vs time profiles for the 60 mg dose and the mean of the three 80 mg doses for individual subjects administered an oral formulation of the present invention.

### SUMMARY OF THE INVENTION

In a first embodiment, the present invention comprises a controlled release pharmaceutical composition for oral administration for enhanced systemic delivery of a cytidine analog comprising a therapeutically effective amount of a cytidine analog and a drug release controlling component which is capable of providing release of the cytidine analog primarily in the large intestine. After ingestion by a patient, the cytidine analog is released primarily in the large intestine.

In another embodiment, the present invention includes a method for treating a patient having a disease associated with abnormal cell proliferation. The method includes orally administering to the patient a controlled release pharmaceutical composition, comprising a therapeutically effective amount of a cytidine analog and a drug release controlling component which is capable of providing release of the cytidine analog primarily in the large intestine. After ingestion by a patient the cytidine analog is released primarily in the large intestine.

In another embodiment, the present invention includes a method of increasing the bioavailability of a cytidine analog upon administration to a patient, comprising the following steps. First, provided is a controlled release pharmaceutical composition, comprising a therapeutically effective amount of a cytidine analog and a drug release controlling component capable of providing release of the cytidine analog primarily in the large intestine. Second, the patient ingests the composition, whereupon the composition contacts the biological fluids of the patient's body and increases the bioavailability of the cytidine analog.

In one embodiment, a condition to treat using the present invention is a myelodysplastic syndrome. In one embodiment, the cytidine analog is 5-azacytidine. In one embodiment, the drug release controlling component is an enteric coating.

### DETAILED DESCRIPTION OF THE INVENTION

The present invention is based on the surprising discovery that 5-azacytidine and related compounds are best absorbed in the lower gastrointestinal tract, i.e., the large intestine (colon). Conventionally, it is expected that the upper gastrointestinal tract is the more desirable location for absorption, due to greater surface area, relatively greater liquidity, and the fact that typically the greater part of absorption of nutrients takes place therein. However, the inventors have found that in the case for cytidine analogs, absorption is greatest and most consistent between patients in colonic tissue. Accordingly, the present invention demonstrates the preparation of a solid oral dosage form of a cytidine analog, such as 5-azacytidine, using common pharmaceutical excipients designed for delivering pharmaceutical compositions to the large intestine and colon. The term “absorb”, “absorption”, “absorbed” and the like are used to indicate transfer of a cytidine analog across a relevant tissue, such as, for example, intestinal tissue. In some embodiments, absorbed cytidine analogs are taken up by the blood stream making the cytidine analog available at least partially systemically. In some embodiments, absorption occurs without substantive degradation (i.e., undesirable chemical modification of) of the cytidine analog.

Furthermore, the inventors have demonstrated that inclusion of THU (taught by others as a requirement to facilitate bioavailability of this drug class) is not necessary to achieve useful oral bioavailability of cytidine analogs via delivery in the large intestine and colon. Accordingly, formulations of the present invention obviate the need to utilize enzymatic inhibitors such as THU in formulations to increase bioavailability of cytidine analogs. Avoidance of enzymatic inhibitors is a desirable attribute for a therapeutic dosage form since such inclusion increases the formulation cost and complexity, and may result in instability, or undesirable, pharmacological, toxicological or other effects. Accordingly, oral delivery of 5-azacytidine without inclusion of an enzymatic inhibitor is possible when the target tissue to which the drug is delivered is the colon. In the case of PO delivery of 5-azacytidine to humans, data suggests that delivery to the upper GI tract may well benefit from enzymatic inhibition, however delivery to the colon does not require the inclusion of such an inhibitor. Targeting to the colon may be achieved with commercially available and pharmaceutically acceptable coatings such as, for example, enteric coatings.

Furthermore, the inventors have demonstrated the preparation of solid oral dosage forms containing excipients and coatings which possess acceptable production and stability characteristics for use as a pharmaceutical dosage form.

In one embodiment, the present invention includes a controlled release pharmaceutical composition for oral administration comprising a) a therapeutically effective amount of a cytidine analog and b) a drug release controlling component for providing the release of the cytidine analog primarily in the large intestine. The controlled release pharmaceutical compositions of the present invention will in one embodiment lack THU.

In one embodiment, the cytidine analog useful in the present invention includes any moiety which is structurally related to cytidine or deoxycytidine and functionally mimics and/or antagonizes the action of cytidine or deoxycytidine. These analogs may also be called cytidine derivatives herein. In one embodiment, cytidine analogs to use with the present invention include 5-aza-2′-deoxycytidine (decitabine), 5-azacytidine, 5-aza-2′-deoxy-2′,2′-difluorocytidine, 5-aza-2′-deoxy-2′-fluorocytidine, 2′-deoxy-2′,2′-difluorocytidine (also called gemcitabine), or cytosine 1-β-D-arabinofuranoside (also called ara-C), 2(1H) pyrimidine riboside (also called zebularine), 2′-cyclocytidine, arabinofuanosyl-5-azacytidine, dihydro-5-azacytidine, N⁴-octadecyl-cytarabine, and elaidic acid cytarabine. In one embodiment, is 5-azacytidine and 5-aza-2′-deoxycytidine The definition of cytidine analog used herein also includes mixtures of cytidine analogs.

Cytidine analogs useful in the present invention may be manufactured by any methods known in the art. In one embodiment, methods to manufacture include methods as disclosed in U.S. Ser. No. 10/390,526 (U.S. Pat. No. 7,038,038); U.S. Ser. No. 10/390,578 (U.S. Pat. No. 6,887,855); U.S. Ser. No. 11/052,615 (U.S. Pat. No. 7,078,518); U.S. Ser. No. 10,390,530 (U.S. Pat. No. 6,943,249); and U.S. Ser. No. 10/823,394, all incorporated by reference herein in their entireties.

In one embodiment, the amounts of a cytidine analog to use in methods of the present invention and in the oral formulations of the present invention include a therapeutically effective amount. Therapeutic indications are discussed more fully herein below. Precise amounts for therapeutically effective amounts of the cytidine analog in the pharmaceutical compositions of the present invention will vary depending on the age, weight, disease and condition of the patient. For example, pharmaceutical compositions may contain sufficient quantities of a cytidine analog to provide a daily dosage of about 150 mg/m² (based on patient body surface area) or about 4 mg/kg (based on patient body weight) as single or divided (2-3) daily doses.

The controlled release pharmaceutical compositions of the present invention include a drug release controlling component. The drug release controlling component is adjusted such that the release of the cytidine analog occurs primarily in the large intestine. In one embodiment, at least about 95% of the cytidine analog is released in the large intestine, or at least about 90% of the cytidine analog is released in the large intestine. In other embodiments, at least about 80% of the cytidine analog is released in the large intestine, at least about 70% of the cytidine analog is released in the large intestine, at least about 60% of the cytidine analog is released in the large intestine, or at least about 50% of the cytidine analog is released in the large intestine. In other embodiments, the amount released in the intestines is at least about 40%, at least about 30%, or at least about 20% of the cytidine analog. The term “release” refers to the process whereby the cytidine analog is made available for uptake by or transport across the epithelial cells that line the large intestine and is made available to the body.

The pharmaceutical compositions of the present invention are intended for oral delivery. Oral delivery includes formats such as tablets, capsules, caplets, solutions, suspensions and/or syrups, and may also comprise a plurality of granules, beads, powders or pellets that may or may not be encapsulated. Such formats may also be referred to as the “drug core” which contains the cytidine analog. Such dosage forms are prepared using conventional methods known to those in the field of pharmaceutical formulation and are described in the pertinent texts, e.g., in REMINGTON: THE SCIENCE AND PRACTICE OF PHARMACY, 20th Edition, Lippincott Williams & Wilkins, 2000).

Tablets and capsules represent the most convenient oral dosage forms, in which case solid pharmaceutical carriers are employed. Tablets are used in one embodiment. Tablets may be manufactured using standard tablet processing procedures and equipment. One method for forming tablets is by direct compression of a powdered, crystalline or granular composition containing the cytidine analog, alone or in combination with one or more carriers, additives, or the like. As an alternative to direct compression, tablets can be prepared using wet-granulation or dry-granulation processes. Tablets may also be molded rather than compressed, starting with a moist or otherwise tractable material; particularly, compression and granulation techniques are used in one embodiment.

In another embodiment, capsules may be used. Soft gelatin capsules may be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin. A hard gelatin capsule shell can be prepared from a capsule composition comprising gelatin and a small amount of plasticizer such as glycerol. As an alternative to gelatin, the capsule shell may be made of a carbohydrate material. The capsule composition may additionally include colorings, flavorings and opacifiers as required.

The cytidine analog in one embodiment is prepared as a controlled release tablet or capsule which includes a drug core comprising the pharmaceutical composition and optional excipients (described elsewhere herein). Optionally, a “seal coat”, described elsewhere herein, is applied to the drug core before addition of the drug release component. The drug release component is formulated to provide for release of the cytidine analog primarily in the large intestine (colon). In one embodiment, minimal release of the cytidine analog occurs in the upper reaches of the gastrointestinal tract, e.g., the stomach and small intestine.

The small intestine extends from the pylorus to the colic valve where it ends in the large intestine. The small intestine is about 6 meters long and is divisible into three portions: the duodenum, the jejunum, and the ileum. The small intestine is especially adapted for transport and absorption of nutrients and other molecules from ingested material, passing through the lining of the small intestine into the blood. The surface cells of the small intestine are highly specialized for digestion and absorption of nutrients. Almost all the body's nutrient absorption occurs in the small intestine, along its three sub-divisions: the duodenum, jejunum, and ileum. Sites for absorption of specific nutrients (eg: iron, vitamin.B12) are located in these divisions, but most absorption occurs in the jejunum (middle section). Specialized cells contain digestive enzymes, carrier proteins and other secretions. Blood vessels transport nutrients away from the intestine to the liver in the first instance.

Indigestible food passes into the large intestine. By the time ingested material leaves the small intestine, virtually all nutrient absorption will have occurred. The large intestine extends from the end of the ileum (distal ileum) to the anus. The large intestine is divided into the cecum, colon, rectum, and anal canal. The colon is divided into four parts: the ascending, transverse, descending, and sigmoid. The substantial release of the cytidine compound of the present invention may occur in any portion of the large intestine. In one embodiment, release primarily occurs at the upper regions of the large intestine, such as, for example, at the distal ileum, cecum, and/or the ascending colon.

It is known that there are major variations in acidity in the gastrointestinal tract. The stomach is a region of high acidity (about pH 1 to 3). Specific glands and organs emptying into the small intestine raise the pH of the material leaving the stomach to approximately pH 6.0 to 6.5. The large intestine and the colon are about pH 6.4 to 7.0. The transit time through the small intestine is approximately three hours. In contrast, the transit time through the large intestine is approximately 35 hours.

Methods by which to formulate compositions to target specific regions of the gastrointestinal tract are known in the art, described in numerous publications, and all references specifically cited within the present document are incorporated by reference herein. For example, release of drug in the gastrointestinal tract may be accomplished by choosing a drug release controlling component to work together with some physical, chemical or biochemical process in the gastrointestinal tract. A drug release controlling component may take advantage of processes and/or conditions within the gastrointestinal tract and in specific regions of the gastrointestinal tract such as, for example, osmotic pressure, hydrodynamic pressure, vapor pressure, mechanical action, hydration status, pH, bacterial flora, and enzymes. Specific U.S. patents incorporated by reference herein include, among others, U.S. Pat. No. 3,952,741, U.S. Pat. No. 5,464,633, U.S. Pat. No. 5,474,784, U.S. Pat. No. 5,112,621.

Optionally, pharmaceutical compositions of the present invention including drug cores may further comprise a seal coating material that seals the drug to prevent decomposition due to exposure to moisture, such as hydroxypropylmethylcellulose. Accordingly, the drug core of the pharmaceutical composition (containing the cytidine analog) may first be sealed with the seal coating material and then coated with the drug release controlling component to prevent decomposition of the cytidine analog by exposure to moisture. Seal coating materials include, in one embodiment, acetyltributyl citrate, acetyltriethyl citrate, calcium carbonate, carauba wax, cellulose acetate, cellulose acetate phthalate, cetyl alcohol, chitosan, ethylcellulose, fructose, gelatin, glycerin, glyceryl behenate, glyceryl palmitostearate, hydroxyethyl cellulose, hydroxyethylmethyl cellulose, hydroxypropyl cellulose, hypromellose, hypromellose phthalate, isomalt, latex particles, maltitol, maltodextrin, methylcellulose, microcrystalline wax, paraffin, poloxamer, polydextrose, polyethylene glycol, polyvinyl acetate phthalate, polyvinyl alcohol, povidone, shellac, shellac with stearic acid, sodium carboxymethyl cellulose, sucrose, titanium oxide, tributyl citrate, triethyl citrate, vanillin, white wax, xylitol, yellow wax, and zein. Compositions of the present invention may also include film forming agents, which include, for example, ammonium alginate, calcium carbonate, chitosan, chlorpheniramine maleate, copovidone, dibutyl phthalate, dibutyl sebacate, diethyl phthalate, dimethyl phthalate, ethyl lactate, ethylcellulose, gelatin, hydroxyyethyl cellulose, hydroxypropyl cellulose, hypromellose, hypromellose acetate succinate, maltodextrin, polydextrose, polyethylene glycol, polyethylene oxide, polymethylacrylates, poly(methylvinyl ether/maleic anhydride), polyvinylacetate phthalate, triethyl citrate, and vanillin. The amount of seal coating will vary in accordance with factors known by those of skill in the art. The amount of seal coat is, in one embodiment, about 1% w/w of the drug core; about 2%, w/w of the drug core, about 3%, w/w, of the drug core, about 4%, w/w, of the drug core; about 5% w/w of the drug core; about 6%, w/w of the drug core, about 7%, w/w, of the drug core, about 8%, w/w/, of the drug core; about 9% w/w of the drug core; about 10%, w/w of the drug core, about 11%, w/w, of the drug core, about 12%, w/w, of the drug core; about 14% w/w of the drug core; about 16%, w/w of the drug core, about 18%, w/w, of the drug core, about 20%, w/w, of the drug core; or more, if determined to be appropriate. Seal coats may also be applied at amounts between about 1% and about 10% w/w of the drug core, between about 2% and 9% w/w of the drug core, between about 3% and 8% w/w of the drug core, between about 4% and 7% w/w of the drug core, and between about 5% and about 6% w/w of the drug core.

In one embodiment, drug release controlling components include, for example, coatings, matrices, or physical changes. Coatings are used in one embodiment. Coatings include, for example, enteric coatings, time delay coatings, bacterially degradable coatings, and mixtures thereof. The pharmaceutical composition may comprise multiple coatings of either the same or different types of coatings. In choosing an appropriate coating or mixture thereof, the formulations practitioner may consider a number of variables influencing the location in which a drug will become available in the gastrointestinal tract, e.g., the pH at which coatings dissolve; the time of dissolution (which is influenced by thickness of the coatings and/or additional components in the coatings); time of transit through the gastrointestinal tract, and whether the coatings can be degraded by the patent's digestive enzymes or require enzymes present only in bacteria residing in the lower intestine. As an example of a combination drug release controlling component is, for example, an inner core with two polymeric layers. The outer layer, an enteric coating, may be chosen to dissolve at a pH level above 5. The inner layer, may be made up of hydroxypropylmethylcellulose to act as a time delay component to delay drug release for a predetermined period. The thickness of the inner layer can be adjusted to determine the lag time.

Methods by which skilled practitioners can assess where a drug is released in the gastrointestinal tract of either animal models or human volunteers are known in the art, and include scintigraphic studies, testing in biorelevant medium which simulates the fluid in relevant portions of the gastrointestinal tract, among others.

In one embodiment, a drug release controlling component may include an enteric coating. The term “enteric coating” refers to a coating that allows a cytidine analog formulation to pass through the stomach substantially intact and subsequently disintegrate substantially in the intestines. In one embodiment, the disintegration occurs in the large intestine.

The coating of pH-sensitive (enteric) polymers to tablets, capsules and other oral formulations of the present invention provided delayed release and protect the active drug from gastric fluid. In general, enteric coatings should be able to withstand the lower pH values of the stomach and small intestine and be able to disintegrate at the neutral or slightly alkaline pH of the large intestine. Enteric coatings are a well known class of compounds. Coating pharmaceutically active compositions with enteric coatings is well known in the art to enable pharmaceutical compositions to bypass the stomach and its low acidity. Enteric coatings generally refer to a class of compounds that dissolve at or above a particular pH and include a number of pH-sensitive polymers. The pH dependent coating polymer may be selected from those enteric coatings known to those skilled in the art. Such polymers may be one or more of the group comprising hydroxypropylmethylcellulose phthalate, polyvinyl acetate phthalate (PVAP), hydroxypropylmethylcellulose acetate succinate (HPMCAS), alginate, carbomer, carboxymethyl cellulose, methacrylic acid copolymer (such as, for example, a cationic copolymer of dimethyl aminoethyl methacrylate and neutral methacrylic esters), polyvinyl acetate phthalate, cellulose acetate trimellitate, shellac, cellulose acetate phthalate (CAP), starch glycolate, polacrylin, methyl cellulose acetate phthalate, hydroxymethylcellulose phthalate, hydroxymethylmethylcellulose acetate succinate, hydroxypropylcellulose acetate phthalate, cellulose acetate terephthalate, cellulose acetate isophthalate, and includes the various grades of each polymer such as HPMCAS-LF, HPMCAS-MF and HPMCAS-HG, or mixtures thereof. Other enteric coatings suitable for the present invention include acetyltributyl citrate, carbomers, guar gum, hypromellose acetate succinate, hypromellose phthalate, polymethacrylates, tributyl citrate, triethyl citrate, white wax, and zein.

In one embodiment, the pH dependent coating is selected from the group consisting of methacrylic acid copolymers of varying threshold pH (such as, but not limited to EUDRAGIT S 100 (a cationic copolymer of dimethyl aminoethyl methacrylate and neutral methacrylic acid esters manufactured by Rohm Pharma GmbH of Darmstadt, Germany)).

Multiple coatings of enteric polymers may be utilized. In one embodiment, the first coating (closest to the core) is an enteric coating that will survive until the dosage form arrives at the large intestine/colon. To target the large intestine, in one embodiment an enteric coating comprises a series of methacrylic acid anionic copolymers known as EUDRAGIT S. The EUDRAGIT S films are colorless, transparent and brittle. In one embodiment, the enteric coating comprises EUDRAGIT S100. The EUDRAGIT S coatings are insoluble in pure water, in buffer solutions below a pH of 6.0 and also in natural and artificial gastric juices. They are slowly soluble in the region of the digestive tract where the juices are neutral to weakly alkaline (i.e., the large intestine and the colon) and in buffer solutions above a pH of 7.0. Mixtures of these various enteric polymers recited above, can be used in the present invention. Further, the use of plasticizers is included in one embodiment with the enteric polymer coatings useful herein.

As known in the art and discussed in sources such as Patel et al. “Colon Specific Delivery” Drug Delivery Technology (2006) Vol. 6 62-71, and Khan et al., J. Controlled Release 1999; 58:215-222, the disintegration rates of enteric coated tablets are dependent on the polymer combination used to coat the tablets, the pH of the disintegration media, and the coating level of the tablets (i.e., thickness of the coating). The presence of plasticizer and the nature of the salts in the dissolution medium also influence the dissolution rate. A number of specific formulations effective for release in the colon in human volunteers, using in vivo scintigraphic studies, is disclosed in Patel et al., and are incorporated by reference herein.

The enteric coating may also be modified through the inclusion of an edible acid to retard or slow the dissolution of the coating in the intestines. Any edible acid may be used. Representative edible acids include acetic acid, benzoic acid, fumaric acid, sorbic acid, propionic acid, hydrochloric acid, citric acid, malic acid, tartaric acid, isocitric acid, oxalic acid, lactic acid, the phosphoric acids and mixtures thereof. One embodiment includes fumaric acid and malic acids. The weight percent of the edible acid in the enteric coating solution (polymer, plasticizer, anti-tack agents, water and the like) can range from about 5 to about 40%, with 10 to 30% present in one embodiment and 10 to 25% in another embodiment. Those skilled in the art will readily be able to determine the exact amount of edible acid to include in the coating solution, depending upon the pKa of the particular edible acid and the desired delay in dissolution of the enteric coating. After application of the enteric coating solution, as further described below, the percent of edible acid in the coating will range from about 10 to about 80 weight % of the coating; 20 to 60% in one embodiment; and 25-50% in another.

Enteric coatings can be obtained from a number of manufacturers, such as, for example, Rohm Pharma GmbH of Darmstadt, Germany (EUDRAGIT). Particular blends of pH sensitive polymers and types can be selected by one of skill in the art. As an example, the manufacturer of EUDRAGIT polymers teaches that the EUDRAGIT grades for sustained release formulations are based on copolymers of acrylate and methacrylates with quaternary ammonium groups as functional groups as well as ethylacrylate methylmethacrylate copolymers with a neutral ester group. EUDRAGIT polymers are available insoluble and/or permeable. For example, the EUDRAGIT RL-types are highly permeable, the EUDRAGIT RS-types are poorly permeable, the EUDRAGIT NE-types are swellable and permeable. The release profiles and locations of release can be determined by varying mixing ratios of the polymers and/or film thickness of the coatings and such profiles can be adjusted by those of skill in the art.

In some embodiments, coatings include those that selectively dissolve at a pH at or above the pH generally prevailing in the large intestine, for example, above about pH 6, above about pH 6.2, above about pH 6.4, above about pH 6.6, above about pH 6.8, or above about pH 7. In one embodiment, the enteric coating will selectively dissolve in the pH range of about 6.0 to about 7.5, in the pH range of about 6.2 to about 7.5, in the pH range of about 6.4 to about 7.2, in the pH range of about 6.5 to about 7, in the pH range of about 6.5 to 6.8. As an example of coatings and their “threshold” pH (the pH at which the coating will dissolve) which the skilled practitioner may consider include, but are not limited to, cellulose phthalates (e.g, hydropropylmethylcellulose phthalates (HPMCPs)) that selectively dissolve at pH above 5.6, the EUDRAGIT family of polymers which are anionic polymer based on methacrylic acid and methacrylates with carboxyl functional groups (e.g., EUDRAGIT L30D with threshold pH of 5.6, EUDRAGIT L with threshold pH of 6.0, and EUDRAGIT S with threshold pH of 6.8), AQUATERIC with threshold pH of 5.8, polyvinylacetate phthalate (PVAP) that releases drug at pH values above about 5.0, shellac that is obtained from a gummy exudation produced by female insects, Laccifer lacca kerr, and releases drug at about pH 7.0, and cellulose acetate phthalate (CAP) with threshold pH of 6.0. In a one embodiment, the drug is enteric-coated with EUDRAGIT S100 with threshold pH of 7.0, which will degrade measurably at slightly lower pH such as pH 6.8.

In one embodiment, prior to application to the tablets, capsules, or drug core of the present invention, the drug release controlling component, such as, for example, the enteric coatings useful in the present invention, will be dissolved in a non-aqueous solution in order to create the solid oral formulation of the present invention. Examples of such non aqueous solutions include any known in the art suitable for pharmaceutical formulation procedures, including, for example, acetone-isopropanol solvent mixtures, methylene chloride-ethanol solvent mixtures, acetone-ethanol solvent mixtures, benzene-methanol solvent mixtures, acetate-ethanol solvent mixtures, among others. Proportions of each solvent to use and conditions will be readily determined by those of skill in the art. The solid dispersion of the composition of the present invention, in one embodiment, can be formed by spray drying techniques, although it will be understood that suitable solid dispersions may be formed by a skilled addressee utilizing other conventional techniques, such as co-grinding, melt extrusion, freeze drying, rotary evaporation or any solvent removal process. In one embodiment, spray drying is utilized. The enteric coating may be applied over the entire surface area or portions thereof. In one embodiment, the entire surface area is coated.

In one embodiment, the enteric coat comprises EUDRAGIT S100 and the amount of enteric coat to use, relative to the drug core, or additional to the drug core, an amount of about 1% w/w of the drug core, about 2% w/w of the drug core; about 3% w/w of the drug core; about 4%, w/w of the drug core, about 5% w/w of the drug core; about 6%, w/w, of the drug core, about 7% w/w of the drug core, about 8%, w/w, of the drug core; about 9% w/w of the drug core, about 10% w/w of the drug core; about 12%, w/w of the drug core; about 14%, w/w, of the drug core, about 16%, w/w/, of the drug core; about 18% w/w of the drug core; about 20%, w/w of the drug core, about 22%, w/w, of the drug core, about 24%, w/w, of the drug core; about 26% w/w of the drug core; about 28%, w/w of the drug core, about 30%, w/w, of the drug core, about 32%, w/w, of the drug core; about 34% w/w of the drug core; about 36%, w/w of the drug core, about 38%, w/w, of the drug core, about 40%, w/w, of the drug core; about 42% w/w of the drug core; about 44%, w/w of the drug core; about 46%, w/w, of the drug core, about 48%, w/w/, of the drug core; about 50% w/w of the drug core; about 52%, w/w of the drug core, about 54%, w/w, of the drug core, about 56%, w/w, of the drug core; about 58% w/w of the drug core; about 60%, w/w of the drug core, about 62%, w/w, of the drug core, about 64%, w/w, of the drug core; about 66% w/w of the drug core; about 68%, w/w of the drug core, about 70%, w/w, of the drug core, about 72%, w/w, of the drug core; about 74% w/w of the drug core; about 76%, w/w of the drug core; about 78%, w/w, of the drug core, about 80%, w/w/, of the drug core; about 82% w/w of the drug core; about 84%, w/w of the drug core, about 86%, w/w, of the drug core, about 88%, w/w, of the drug core; about 90% w/w of the drug core; about 92%, w/w of the drug core, about 91%, w/w, of the drug core, about 96%, w/w, of the drug core; about 98%, w/w, of the drug core, or more, if determined to be appropriate. Ranges include between about 2% and about 20% w/w additional; between about 3% and about 15% w/w additional; between about 4% and about 10% w/w additional; between about 5% and about 9% w/w additional; between about 6% and about 8% w/w additional.

As referenced elsewhere herein, enteric coats (and any drug release controlling component of the present invention, including time delay and bacterially degradable coats, and mixtures thereof) may also optionally further comprise a plasticizer. If a plasticizer is present, the drug release controlling component may be present in an amount of about 1% w/w of the coat, about 2% w/w of the coat; about 3% w/w of the coat; about 4%, w/w of the coat, about 5% w/w of the coat; about 6%, w/w, of the coat, about 7% w/w of the coat, about 8%, w/w, of the coat; about 9% w/w of the coat, about 10% w/w of the coat; about 12%, w/w of the coat; about 14%, w/w, of the coat, about 16%, w/w/, of the coat; about 18% w/w of the coat; about 20%, w/w of the coat, about 22%, w/w, of the coat, about 24%, w/w, of the coat; about 26% w/w of the coat; about 28%, w/w of the coat, about 30%, w/w, of the coat, about 32%, w/w, of the coat; about 34% w/w of the coat; about 36%, w/w of the coat, about 38%, w/w, of the coat, about 40%, w/w, of the coat; about 42% w/w of the coat; about 44%, w/w of the coat; about 46%, w/w, of the coat, about 48%, w/w/, of the coat; about 50% w/w of the coat; about 52%, w/w of the coat, about 54%, w/w, of the coat, about 56%, w/w, of the coat; about 58% w/w of the coat; about 60%, w/w of the coat, about 62%, w/w, of the coat, about 64%, w/w, of the coat; about 66% w/w of the coat; about 68%, w/w of the coat, about 70%, w/w, of the coat, about 72%, w/w, of the coat; about 74% w/w of the coat; about 76%, w/w of the coat; about 78%, w/w, of the coat, about 80%, w/w/, of the coat; about 82% w/w of the coat; about 84%, w/w of the coat, about 86%, w/w, of the coat, about 88%, w/w, of the coat; about 90% w/w of the coat; about 92%, w/w of the coat, about 91%, w/w, of the coat, about 96%, w/w, of the coat; about 98%, w/w, of the coat, or more, if determined to be appropriate. Ranges include between about 30% and about 95% w/w of the coat; between about 40% and about 95% w/w of the coat; between about 50% and about 95% w/w of the coat; between about 60% and about 95% w/w of the coat; between about 70% and about 95% w/w coat.

In another embodiment, the amount of the enteric-coating material, when the coating material is EUDRAGIT S100, in one embodiment is about 1-10% w/w in the total composition, about 3-8% w/w in the total composition, or 4-7% w/w in the total composition. Appropriate amounts of coating to use will vary depending on the type of coating used, tablet size, surface preparation, target dissolution time at a given pH, etc. All things being equal, to determine the amount and/or thickness of the enteric coating, as the pH threshold of the enteric coating material increases, the relative amount and thickness of the coating can be decreased to achieve dissolution of the tablet in a specified time frame at a particular pH. Routine empirical studies to determine the optimum conditions for targeting the large intestine may be carried out by the skilled person.

According to the invention, the controlled release pharmaceutical composition comprising a cytidine analog and an enteric coating, in one embodiment, will not substantially disintegrate in an acidic, aqueous medium at about pH 1-3 for at least one hour, at least two hours, or at least three hours. The composition is considered to be substantially disintegrated if at least 50% of the composition disintegrates, e.g., undergoes rupture and release. In some embodiments, the controlled release pharmaceutical composition comprising a cytidine analog and an enteric coating disintegrates substantially in an aqueous medium at about pH 7 or above within about ten hours, within about eight hours, within about six hours, within about four hours, within about two hours, within about one hour, within about 45 minutes, within about 30 minutes, and within about 15 minutes. In some embodiments, the controlled release pharmaceutical composition comprising a cytidine analog and an enteric coating disintegrates substantially in an aqueous medium at about pH 6.8 or above within about ten hours, within about eight hours, within about six hours, within about four hours, within about two hours, within about one hour, within about 45 minutes, within about 30 minutes, and within about 15 minutes. In some embodiments, times are within about two hours or less. In some embodiments, the controlled release pharmaceutical composition comprising a cytidine analog and an enteric coating does not disintegrate substantially in an aqueous medium at about pH 5 to about pH 6.5 for at least one hour, for at least about 1.5 hours, for at least about two hours, for at least about 2.5 hours, for at least about 3 hours, for at least about 3.5 hours, or for at least about four hours.

According to the invention, the controlled release pharmaceutical composition comprising a cytidine analog and an enteric coating, in one embodiment, will not substantially disintegrate after ingestion by a patient, on average, for at least about one hour, at least two hours, at least about three hours, at least about four hours, at least about five hours, at least about six hours, at least about seven hours, at least about eight hours, at least about nine hours, at least about ten hours, at least about twelve hours, at least about fourteen hours, at least about sixteen hours, at least about eighteen hours, at least about twenty hours, at least about twenty four hours, at least about twenty eight hours, or at least about thirty two hours. According to the invention, the controlled release pharmaceutical composition comprising a cytidine analog and an enteric coating, in some embodiments, will substantially disintegrate after ingestion by a patient within about three hours, within about four hours, within about five hours, within about six hours, within about seven hours, within about eight hours, within about nine hours, within about ten hours, within about twelve hours, within about fourteen hours, within about sixteen hours, within about eighteen hours, within about twenty hours, within about twenty two hours, within about twenty four hours, within about twenty six hours, within about twenty eight hours, or within about thirty hours. The composition is considered to be substantially disintegrated if at least 50% of the composition disintegrates, e.g., undergoes rupture and release.

As examples of more specific types of controlled release pharmaceutical compositions, the following is described. For example, a pharmaceutical composition comprising a solid oral dosage form may be coated with a coating of a 60 to 150 micrometer thick layer of an anionic polymer which is insoluble in gastric juice and in intestinal fluid below pH 7 but soluble in colonic intestinal juice, so that the dosage form remains intact until the colon. For example, a pharmaceutical composition may be coated with a material which dissolves in the intestine and contained within a capsule which is also coated with a material which dissolves in the intestine. The composition is for selectively administering the drug to the intestine. The granules are in one embodiment contained within an enterically coated capsule which releases the granules in the small intestine and that the granules are coated with a coating which remains substantially intact until they reach at least the ileum and in one embodiment, thereafter provide a sustained or immediate release of the drug through the colon. Also there may be a non-disintegratable solid enteric pharmaceutical composition comprising a cytidine analog having relatively rapid dissolution at pH 6.5 from an excipient matrix, and dosage forms containing pellets of the composition. The rapid dissolution is increased by the presence of a rheological modifying super-disintegrant in an amount of at least 5% by weight, but insufficient to cause disintegration of the composition. It is stated that the composition may comprise a plurality of such pellets, which may be coated in an enteric coating such as cellulose acetate phthalate or, partly methyl esterified methacrylic acid polymers having a ratio of free acid groups to ester groups of about 1:2, contained in a capsule that is enterically coated with a suitable coating material. The coating material on the pellets is in one embodiment, one that is insoluble in gastric juices and intestinal fluid below pH 7, but is soluble in the lower intestine. The enteric coating material of the capsule is chosen to protect the capsule during passage through the stomach.

In another embodiment, the pharmaceutical compositions of the present invention may include time delay coatings to delay release of the cytidine analog until reaching the large intestine. In accordance with this embodiment, solid dosage forms, whether tablets, capsules, caplets, or particulates, may, if desired, be coated so as to provide for delayed release. Sustained release dosage forms provide for drug release over an extended time period, and may or may not be delayed release. Generally, as will be appreciated by those of ordinary skill in the art, sustained release dosage forms are formulated by either dispersing a drug within a matrix of, a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene, vinyl polymers and copolymers such as polyvinyl pyrrolidone, polyvinyl acetate, polyvinylacetate phthalate, vinylacetate crotonic acid copolymer, and ethylene-vinyl acetate copolymers, zein, and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

In particular, time release coatings useful in the present invention may include the following: acetyltributyl citrate, acetyltriethyl citrate, aliphatic polyesters, bentonite, carbomers, carrageenan, cellulose acetate, cellulose acetate phthalate, ceratonia, cetyl alcohol, cetyl esters wax, chitosan, dibutyl sebacate, ethylcellulose, glycerin monostearate, glyceryl behenate, glyceryl monooleate, glyceryl monostearate, glyceryl palmitostearate, guar gum, hydroxypropyl cellulose, hypromellose acetate succinate, isopropyl palmitate, magnesium aluminum silicate, magnesium oxide, methylcellulose, microcrystalline wax, paraffin, peanut oil, potassium polacrilin, polycarbophil, polyethylene oxide, polymethacrylates, povidone, stearic acid, stearyl alcohol, talc, tributyl citrate, triethyl citrate, white wax, xanthan gum, yellow wax, and zein. Another useful time release coating is EUDRAGIT RS PO, copolymers of acrylic andmethacrylic acid esters with a low content in quaternary ammonium groups with an average molecular weight of about 150,000 D. This polymer has low permeability and water solubility with swelling that is pH-independent.

The type, amount and/or thickness of the matrix or coating can be readily adjusted by one of skill in the art to obtain the desired release profiles and timing. In one embodiment, the time release coating is EUDRAGIT RS PO, and the coating will vary in accordance with factors known by those of skill in the art. The amount of time release coat is, in one embodiment, about 1% w/w of the drug core; about 2%, w/w of the drug core, about 3%, w/w, of the drug core, about 4%, w/w, of the drug core; about 5% w/w of the drug core; about 6%, w/w of the drug core, about 7%, w/w, of the drug core, about 8%, w/w/, of the drug core; about 9% w/w of the drug core; about 10%, w/w of the drug core, about 11%, w/w, of the drug core, about 12%, w/w, of the drug core; about 14% w/w of the drug core; about 16%, w/w of the drug core, about 18%, w/w, of the drug core, about 20%, w/w, of the drug core; or more, if determined to be appropriate.

In one embodiment, the controlled release formulation comprising a time delay component does not allow substantial release of the cytidine analog for at least about three hours after oral ingestion by a patient, for at least about four hours after oral ingestion by a patient, for at least about five hours after oral ingestion by a patient, for at least about six hours after ingestion by a patient, for at least about eight hours after ingestion by a patient, or for at least about ten hours after ingestion by a patient. In one embodiment, a dissolution time period is, in one embodiment, between about three hours and about ten hours, between about four hours and about six hours. A nominal lag time of five hours is usually considered sufficient, since small intestinal transit has been considered relatively constant at about 3 to 4 hours. Multiple coatings of either the same or different types of a time delay release component may be used by one of skill in the art to target the large intestine and/or colon.

Examples of time delay methods are known in the art, and include PULSINCAP device, which consists of a non-disintegrating half capsule shell sealed at the open end with a hydrogel plug. The plug hydrates on contact with gastrointestinal fluid, and swells to an extend that it is expelled from the capsule body, thus releasing the drug. Usually, the time it takes the hydrogel plug to hydrate and eject from the capsule shell defined the lag time prior to drug release, and hence, by altering the composition and size of the hydrogel plug, it is possible to achieve drug release after varying lag times. As another example, a pulsed system, called the TIME CLOCK SYSTEM, comprises a solid dosage form coating with a hydrophobic surfactant layer to which a water-soluble polymer is attached to improve adhesion to the core. The thickness of the outer layer determines the time required to disperse in an aqueous environment. After dispersion of the outer layer, the core becomes available for dispersion.

In another embodiment of the present invention, the pharmaceutical compositions may include a bacterially degradable component, such as a coating, in order to delay release of the cytidine analog until reaching the large intestine. For example, the digestive excretions in the human gastrointestinal tract lack specific enzymes that can degrade certain types of oligosaccharides, such as, for example, cellulose. In contrast, bacteria existing at the level of the large intestine have the ability to digest many of these types of polysaccharides. Accordingly, a coating or matrix which undergoes bacterial degradation in the colonic surroundings and dissolves/degrades causing release of its drug content is compatible with the present invention. A number of flora are typically found in the human gastrointestinal tract. The flora may change depending upon the physiological condition of the person or animal being treated. Drug delivery may be designed to specifically target a type of flora known to be in abundance in a patient or a population of patients.

A partial list of oligosaccharides suitable for incorporation into the controlled release pharmaceutical compositions of the present invention, in one embodiment, includes those which can be digested by colonic bacteria but not by the enzymes present at the level of a patient's stomach or small intestine. Examples of such oligosaccharides are cellobiose, lactulose, the trisaccharide raffinose and stachyose and polymers thereof, such as cellulose. Oligosaccharides also include amylose, arabinogalactan, chitosan, chondroitin, cyclodextrin, dextran, guar gum, inulin, pectin, and xylan. Natural polymers such as mucopolysaccharides can also be the basis of bacterially degradable coatings. Most of these natural polymers are, in their unmodified form, soluble in water and gastric fluid. Accordingly, in one embodiment, natural polymers are cross linked to reduce the hydrophilicity of these polymers and thus allow their utility in the compositions and methods of the invention as colonic drug carriers which pass the small intestine and degrade in the colon. Accordingly, bacterially degradable components are, in one embodiment, covalently or non covalently bonded to a polymer, or mixed with a polymer in one embodiment an acrylic polymer, including, a methacrylic polymer, such as a EUDRAGIT polymer.

A non-limiting example of one cross-linking method is amide protection by the reaction of diamine with the polymer. Diamines that can be used include: 1,4 butanediamine, 1,6 hexanediamine, 1,7 heptanediamine and 1,12 dodecanediamine. A number of U.S. patents and patent application publications discuss methods by which to control release of drugs via bacterially degradable coatings and/or matrices, such as, for example, U.S. Pat. No. 5,525,634, U.S. Pat. No. 5,849,327, U.S. Pat. No. 4,432,966, U.S. Pat. No. 5,112,621, and U.S. Pat. No. 5,536,507, all of which are incorporated by reference herein in their entireties.

In one embodiment, the oligosaccharides are formulated together with a seal coat or time release component. In one embodiment, the oligosaccharide is a mixture of chitosan and pectin, in any ratio, for example, 1:1. In another embodiment, the oligosaccharide is amylose. In one embodiment, oligosaccarides and/or mixtures of oligosaccharides are formulated together with EUDRAGIT RS PO. For example, the oligosaccharide or mixture thereof can be present in a bacterially degradable coat in the amount of about 2% w/w of the coat; about 4%, w/w of the coat, about 6%, w/w, of the coat, about 8%, w/w, of the coat; about 10% w/w of the coat; about 12%, w/w of the coat; about 14%, w/w, of the coat, about 16%, w/w/, of the coat; about 18% w/w of the coat; about 20%, w/w of the coat, about 22%, w/w, of the coat, about 24%, w/w, of the coat; about 26% w/w of the coat; about 28%, w/w of the coat, about 30%, w/w, of the coat, about 32%, w/w, of the coat; about 34% w/w of the coat; about 36%, w/w of the coat, about 38%, w/w, of the coat, about 40%, w/w, of the coat; about 42% w/w of the coat; about 44%, w/w of the coat; about 46%, w/w, of the coat, about 48%, w/w/, of the coat; about 50% w/w of the coat; about 52%, w/w of the coat, about 54%, w/w, of the coat, about 56%, w/w, of the coat; about 58% w/w of the coat; about 60%, w/w of the coat, about 62%, w/w, of the coat, about 64%, w/w, of the coat; about 66% w/w of the coat; about 68%, w/w of the coat, about 70%, w/w, of the coat, about 72%, w/w, of the coat; about 74% w/w of the coat; about 76%, w/w of the coat; about 78%, w/w, of the coat, about 80%, w/w/, of the coat; about 82% w/w of the coat; about 84%, w/w of the coat, about 86%, w/w, of the coat, about 88%, w/w, of the coat; about 90% w/w of the coat; about 92%, w/w of the coat, about 91%, w/w, of the coat, about 96%, w/w, of the coat; about 98%, w/w, of the coat, or more, if determined to be appropriate. The balance, in this embodiment, is time release component, for example, EUDRAGIT RS PO, and optionally, plasticizer, each in amounts noted elsewhere herein.

The presence of microbial anaerobic organisms is known to provide reducing conditions in the large intestine and colon. Thus, the coating may also suitably comprise a material that is redox sensitive. Such coatings typically consist of azo-polymers, which can, for example, be composed of a random co-polymer of styrene and hydroxyethyl methacrylate cross-linked with divinyl azo-benzene synthesized by free radical polymerization. The azo-polymer is broken down enzymatically and specifically in the colon delivery system. When drugs are coated with these polymers, they are protected against gastric and intestinal enzymes. The drugs are subsequently released in the large intestine where enzyme activity is low and the azo bond is broken by only microbial enzymes in the colon.

In vitro evaluation of azo-containing polysaccharide gels, more specifically azo-inulin and azo-dextran gels, for colonic delivery has been shown that in vitro azo-polysaccharide gels can be degraded through both reduction of the azo group in the cross-links as well as enzymatic breakdown of the polysaccharide backbone. The azo-polysaccharide gels were synthesized by radical cross-linking of a mixture of methacrylated inulin or methacrylated dextran and NN-bis-(methacryloylamino) azo-benzene (B(MA)AB), and were characterized by dynamic mechanical analysis and swelling measurements. Azo-dextran gels could be obtained from methacrylated dextran having low degrees of substitution but not from minimally substituted methacrylated inulin. Increasing the amount of B(MA)AB resulted in denser azo-inulin and azo-dextran networks. Compared with their swelling in dimethyl formamide, all azo-dextran gels became more swollen in water while azo-inulin gels shrank upon exposure to water, indicating a more hydrophobic character of the azo-inulin gels. Breakdown of the inulin and dextran chains by inulinase and dextranase, respectively, were observed. In one embodiment, the azo-polymer is a polymer of 2-hydroxyethyl methacrylic acid cross linked with divinyl azobenzene (tradename HEMA-DVAB polymer).

In one embodiment, the azo polymer is 2-hydroxyethyl methacrylic acid cross linked with divinyl azobenzene (HEMA-DVAB polymer). The amount of azopolymer can be determined by one of skill in the art, and in one embodiment, is present in a bacterially degradable coat in the amount of about 2% w/w of the coat; about 4%, w/w of the coat, about 6%, w/w, of the coat, about 8%, w/w, of the coat; about 10% w/w of the coat; about 12%, w/w of the coat; about 14%, w/w, of the coat, about 16%, w/w/, of the coat; about 18% w/w of the coat; about 20%, w/w of the coat, about 22%, w/w, of the coat, about 24%, w/w, of the coat; about 26% w/w of the coat; about 28%, w/w of the coat, about 30%, w/w, of the coat, about 32%, w/w, of the coat; about 34% w/w of the coat; about 36%, w/w of the coat, about 38%, w/w, of the coat, about 40%, w/w, of the coat; about 42% w/w of the coat; about 44%, w/w of the coat; about 46%, w/w, of the coat, about 48%, w/w/, of the coat; about 50% w/w of the coat; about 52%, w/w of the coat, about 54%, w/w, of the coat, about 56%, w/w, of the coat; about 58% w/w of the coat; about 60%, w/w of the coat, about 62%, w/w, of the coat, about 64%, w/w, of the coat; about 66% w/w of the coat; about 68%, w/w of the coat, about 70%, w/w, of the coat, about 72%, w/w, of the coat; about 74% w/w of the coat; about 76%, w/w of the coat; about 78%, w/w, of the coat, about 80%, w/w/, of the coat; about 82% w/w of the coat; about 84%, w/w of the coat, about 86%, w/w, of the coat, about 88%, w/w, of the coat; about 90% w/w of the coat; about 92%, w/w of the coat, about 91%, w/w, of the coat, about 96%, w/w, of the coat; about 98%, w/w, of the coat, or more, if determined to be appropriate. The balance, in this embodiment, is time release component, for example, EUDRAGIT RS PO, and optionally, plasticizer, each in amounts noted elsewhere herein.

Below are discussed additional examples of art-known methods of colonic delivery, which incorporate one or more of the methods discussed hereinabove and are suitable for formulating the cytidine compounds. As discussed above, as a general rule, those skilled in the art consider the small intestine to be the preferred target for delivery of acid-labile compounds. Controlled release methods and compositions for targeting or controlling the release of an active compound in the large intestine and colon have been disclosed, including the following. A formulation for site specific release of a cytidine analog in the colon comprises a cytidine analog and amorphous amylose with an outer coating of cellulose or an acrylic polymer material. The active compound is in one embodiment coated with glassy amylose, which tends not to degrade until it reaches the colon where it is attacked by amylose cleaving enzymes provided by microbial flora normally present in the colon. The composition may optionally be further coated with a cellulose or acrylic polymer material, which enhances the delayed release property of the amylose coated composition. The rate of release of the active compound from the composition once it reaches the colon may be controlled by varying the thickness of inner amylose coating provided. It is also possible to vary the release in the colon by coating different particles of the active compound with amylose of different thicknesses. Release characteristics can be further varied by drying, which affects pore size and permeability or by adding a fatty or waxy substance to retard penetration of water. In one embodiment, the cellulose or acrylic polymer outer coating material displays pH independent degradation. Another controlled release composition suitable for delivery of cytidine analog to the colon includes cytidine analog containing spheres also containing microcrystalline cellulose and having diameters in the range 1.00 to 1.40 mm, which spheres are coated with a mixed solvent (water and an organic water miscible solvent) amylose/ethyl cellulose composition, although the latter may instead be an acrylic polymer or hydrophobic coating. Where higher amylose concentrations are present in the coatings, a thicker coating is applied such that release of the cytidine analog should not take place before the colon. Another sustained release pharmaceutical formulation includes coating particles with different thicknesses of a material (such as cellulose acetophthalate) in order to release the drug compound at different rates so as to provide sustained release over a predetermined time period.

Generally, methods for sustained release and/or delivery to the large intestine and/or colon include the following methods disclosed in the following U.S. issued patents, all of which are incorporated herein by reference in their entireties. U.S. Pat. No. 5,529,790 discloses pharmaceutical formulations which provide delayed and sustained release of a drug from the formulation by means of a hydratable diffusion barrier coating. The delay is a consequence of the rate of hydration and the thickness of the coating and the sustained release results from the permeability and thickness of the coating. The diffusion barrier, in one embodiment, consists of a film-forming material that is insoluble in intestinal conditions and at least one further additive which controls the rate of hydration and permeability of the diffusion barrier. One embodiment includes an embodiment where film-forming polymers are non-aqueous or aqueous dispersions of fully esterified acrylic resins (e.g. EUDRAGIT NE30D), fully esterified acrylic resins containing quaternary amine side chains (e.g. EUDRAGIT RS30D) or aqueous dispersions of ethyl cellulose. In one embodiment, an additive for controlling the rate of hydration and the permeability is magnesium stearate. The drug (e.g. diltiazem hydrochloride) may be formulated as spherical microparticles having a diameter in the range 500-1500 micrometers and is, in one embodiment, formulated in two batches of particles, a long delay batch having a low rate of hydration and low permeability and a short delay batch having a relatively high rate of hydration and a high permeability, so that sustained release of the drug can be effected over an extended period of time. Dissolution studies were carried out on particles having different coating thicknesses. U.S. Pat. No. 5,260,069 and U.S. Pat. No. 5,834,024 disclose pharmaceutical compositions comprising at least two pluralities of particles. The pluralities may be coated with different thicknesses of a coating material comprising a polymer blend. The blend comprises, as a major component, at least one water insoluble polymer and, as a minor component, a polymer whose solubility is dependent on pH. U.S. Pat. No. 5,260,069 exemplifies compositions in which nifedipine and zidovudine are active components and U.S. Pat. No. 5,834,024 exemplifies the use of diltiazem as the active component. U.S. Pat. No. 6,267,990 discloses a pharmaceutical composition comprising three pluralities of particles, one of which is uncoated and the other two are coated with different thicknesses of a pH dependent release coating material. U.S. Pat. No. 6,267,990 exemplifies the use of the ACE inhibitor, captopril, as the active component. U.S. Pat. No. 5,834,021 exemplifies a pharmaceutical composition comprising a plurality of pellets comprising prednisolone metasulphobenzoate. The pellets are coated with a first pH dependent release coating material and then filled into a capsule which is then itself coated with a second pH dependent release coating material.

The drug release controlling component may also optionally include a plasticizer. The plasticizer can be added to coating solutions and dispersions to improve the mechanical properties of the polymeric film in the dry state and to influence permeability and drug release when the product is in contact with the release media. Plasticizers can also induce and enhance the coalescence of the colloidal polymer particles in a homogeneous film by reducing the glass transition and minimum film formation temperature (MFT) and to improve the mechanical properties of the dried films. In one embodiment, the plasticizer, by allowing the coating to “flex” slightly, helps to prevent any premature release of the cytidine analog. In one embodiment, the plasticizer is a water-soluble plasticizers, such as, for example, triethyl citrate, triacetin; in another embodiment, the plasticizer is a water-insoluble plasticizers, such as, for example, tributyl citrate, acetyltributyl citrate, acetyltriethylcitrate, dibutyl sebacate, dibutyl phthalate and diethyl phthalate. The plasticizer, if added, may be present at any level known to those of skill in the art to be effective. In some embodiments, the plasticizer is present in the drug release controlling component in an amount of about 2%, about 3%, about 4%, about 5%, about 6%, about 7%, about 8%, about 9%, about 10%, about 11%, about 12%, about 14%, about 16%, about 18%, about 20%, about 25%, w/w, of the coat, or more. In some embodiments, the plasticizer is present in an amount of between about 4% and about 12%, between about 5% and about 11%, between about 6% and about 9%; between about 5% and 40%, between about 5% and 30%, between about 5% and about 20%, w/w, of the coat.

In addition to the cytidine and drug release controlling component, pharmaceutical compositions of the present invention will, in one embodiment, contain one or more other excipients to form a drug “core”. These excipients include diluents (bulking agents), lubricants, disintegrants, fillers, stabilizers, surfactants, preservatives, coloring agents, flavoring agents, binding agents, excipient supports, glidants, permeability enhancement excipients, plasticizers and the like, all of which are known in the art; all named excipients are optional components. It will be understood by those in the art that some substances serve more than one purpose in a pharmaceutical composition. For instance, some substances are binders that help hold a tablet together after compression, yet are disintegrants that help break the tablet apart once it reaches the target delivery site. Selection of excipients and the amounts to use may be readily determined by the formulation scientist based upon experience and consideration of standard procedures and reference works in the field.

Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropylmethylcellulose, methyl cellulose, ethyl cellulose, hydroxyethyl cellulose, carboxymethyl cellulose and the like), veegum, carbomer (e.g. carbopol), sodium, dextrin, guar gum, hydrogenated vegetable oil, magnesium aluminum silicate, maltodextrin, polymethacrylates, povidone (e.g. KOLLIDON, PLASDONE), microcrystalline cellulose, among others. Binding agents also include acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein. The binding agent can be, relative to the drug core, in the amount of about 2% w/w of the drug core; about 4%, w/w of the drug core, about 6%, w/w, of the drug core, about 8%, w/w, of the drug core; about 10% w/w of the drug core t; about 12%, w/w of the drug core; about 14%, w/w, of the drug core, about 16%, w/w/, of the drug core; about 18% w/w of the drug core; about 20%, w/w of the drug core, about 22%, w/w, of the drug core, about 24%, w/w, of the drug core; about 26% w/w of the drug core; about 28%, w/w of the drug core, about 30%, w/w, of the drug core, about 32%, w/w, of the drug core; about 34% w/w of the drug core; about 36%, w/w of the drug core, about 38%, w/w, of the drug core, about 40%, w/w, of the drug core; about 42% w/w of the drug core; about 44%, w/w of the drug core; about 46%, w/w, of the drug core, about 48%, w/w/, of the drug core; about 50% w/w of the drug core; about 52%, w/w of the drug core, about 54%, w/w, of the drug core, about 56%, w/w, of the drug core; about 58% w/w of the drug core; about 60%, w/w of the drug core, about 62%, w/w, of the drug core, about 64%, w/w, of the drug core; about 66% w/w of the drug core; about 68%, w/w of the drug core, about 70%, w/w, of the drug core, about 72%, w/w, of the drug core; about 74% w/w of the drug core; about 76%, w/w of the drug core; about 78%, w/w, of the drug core, about 80%, w/w/, of the drug core; about 82% w/w of the drug core; about 84%, w/w of the drug core, about 86%, w/w, of the drug core, about 88%, w/w, of the drug core; about 90% w/w of the drug core; about 92%, w/w of the drug core, about 91%, w/w, of the drug core, about 96%, w/w, of the drug core; about 98%, w/w, of the drug core, or more, if determined to be appropriate; or between about 5% and about

Diluents are typically necessary to increase bulk so that a practical size tablet is ultimately provided. Suitable diluents include dicalcium phosphate, calcium sulfate, lactose, cellulose, kaolin, mannitol, sodium chloride, dry starch, microcrystalline cellulose (e.g. AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT), potassium chloride, sodium chloride, sorbitol and talc, among others. Diluents also include ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-β-cyclodextrin, talc, tragacanth, trehalose, and xylitol. Generally, diluents are used in amounts calculated to obtain a volume tablet or capsule that is desired; in some embodiments, a diluent is used in an amount of about 5% or more, about 10% or more, about 15% or more, 20% or more, about 22% or more, about 24% or more, about 26% or more, about 28% or more, about 30% or more, about 32% or more, about 34% or more, about 36% or more, about 38% or more, about 40% or more, about 42% or more, about 44% or more, about 46% or more, about 48% or more, about 50% or more, about 52% or more, about 54% or more, about 56% or more, about 58% or more, about 60% or more, about 62% or more, about 64% or more, about 68% or more, about 70% or more, about 72% or more, about 74% or more, about 76% or more, about 78% or more, about 80% or more, about 85% or more, about 90% or more, about 95% or more, weight/weight, of a drug core; between about 10% and about 90%, w/w of the drug core; between about 20% and about 80% w/w of the drug core; between about 30% and about 70% w/w of the drug core; between about 40% and about 60% w/w of the drug core.

Lubricants are used to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. Stearates, if present, in one embodiment represent at no more than approximately 2 wt. % of the drug-containing core. Further examples of lubricants include calcium stearate, glycerin monostearate, glyceryl behenate, glyceryl palmitostearate, magnesium lauryl sulfate, magnesium stearate, myristic acid, palmitic acid, poloxamer, polyethylene glycol, potassium benzoate, sodium benzoate, sodium chloride, sodium lauryl sulfate, sodium stearyl fumarate, stearic acid, talc, and zinc stearate. In one embodiment, the binding agent is magnesium stearate, and is present, relative to the drug core, in the amount of about 0.2% w/w of the drug core; about 0.4%, w/w of the drug core, about 0.6%, w/w, of the drug core, about 0.8%, w/w, of the drug core; about 1.0% w/w of the drug core; about 1.2%, w/w of the drug core; about 1.4%, w/w, of the drug core, about 1.6%, w/w/, of the drug core; about 1.8% w/w of the drug core; about 2.0%, w/w of the drug core, about 2.2%, w/w, of the drug core, about 2.4%, w/w, of the drug core; about 2.6% w/w of the drug core; about 2.8%, w/w of the drug core, about 3.0%, w/w, of the drug core, about 3.5%, w/w, of the drug core; about 4% w/w of the drug core; about 4.5%, w/w of the drug core, about 5%, w/w, of the drug core, about 6%, w/w, of the drug core; about 7% w/w of the drug core; about 8%, w/w of the drug core; about 10%, w/w, of the drug core, about 12%, w/w/, of the drug core; about 14% w/w of the drug core; about 16%, w/w of the drug core, about 18%, w/w, of the drug core, about 20%, w/w, of the drug core; about 25% w/w of the drug core; about 30%, w/w of the drug core, about 35%, w/w, of the drug core, about 40%, w/w, of the drug core; between about 0.2% and about 10%, w/w of the drug core; between about 0.5% and about 5% w/w of the drug core; between about 1% and about 3% w/w of the drug core.

Disintegrants are used to facilitate disintegration of the tablet, and are generally starches, clays, celluloses, algins, gums or crosslinked polymers. Disintegrants also include alginic acid, carboxymethylcellulose calcium, carboxymethylcellulose sodium (e.g. AC-DI-SOL, PRIMELLOSE), colloidal silicon dioxide, croscarmellose sodium, crospovidone (e.g. KOLLIDON, POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB) and starch. Additional disintegrants include alginic acid, calcium alginate, calcium carboxymethylcellulose, chitosan, colloidal silicon dioxide, sodium croscarmellose, crospovidone, sodium docusate, guar gum, hydroxypropyl cellulose, magnesium aluminum silicate, methylcellulose, microcrystalline cellulose, potassium polacrilin, povidone, powdered cellulose, sodium alginate, sodium carboxymethyl cellulose, sodium starch glycolate, starch, and pregelatinized starch. The disintegrant can be, relative to the drug core, in the amount of about 1% w/w of the drug core, about 2% w/w of the drug core; about 3%, w/w/ of the drug core; about 4%, w/w of the drug core; about 5%, w/w/ of the drug core, about 6%, w/w, of the drug core, about 7%, w/w, of the drug core, about 8%, w/w, of the drug core; about 9%, w/w, of the drug core; about 10% w/w of the drug core t; about 12%, w/w of the drug core; about 14%, w/w, of the drug core, about 16%, w/w/, of the drug core; about 18% w/w of the drug core; about 20%, w/w of the drug core, about 22%, w/w, of the drug core, about 24%, w/w, of the drug core; about 26% w/w of the drug core; about 28%, w/w of the drug core, about 30%, w/w, of the drug core, about 32%, w/w, of the drug core; between about 1% and about 10%, w/w of the drug core; between about 2% and about 8% w/w of the drug core; between about 3% and about 7% w/w of the drug core; between about 4% and about 6% w/w of the drug core.

Stabilizers (also called absorption enhancers) are used to inhibit or retard drug decomposition reactions that include, by way of example, oxidative reactions. Stabilizing agents include d-Alpha-tocopheryl polyethylene glycol 1000 succinate (Vitamin E TPGS), acacia, albumin, alginic acid, aluminum stearate, ammonium alginate, ascorbic acid, ascorbyl palmitate, bentonite, butylated hydroxytoluene, calcium alginate, calcium stearate, calcium carboxymethylcellulose, carrageenan, ceratonia, colloidal silicon dioxide, cyclodextrins, diethanolamine, edetates, ethylcellulose, ethyleneglycol palmitostearate, glycerin monostearate, guar gum, hydroxypropyl cellulose, hypromellose, invert sugar, lecithin, magnesium aluminum silicate, monoethanolamine, pectin, poloxamer, polyvinyl alcohol, potassium alginate, potassium polacrilin, povidone, propyl gallate, propylene glycol, propylene glycol alginate, raffinose, sodium acetate, sodium alginate, sodium borate, sodium carboxymethyl cellulose, sodium stearyl fumarate, sorbitol, stearyl alcohol, sulfobutyl-b-cyclodextrin, trehalose, white wax, xanthan gum, xylitol, yellow wax, and zinc acetate. The stabilizer can be, relative to the drug core, in the amount of about 1% w/w of the drug core, about 2% w/w of the drug core; about 3%, w/w/ of the drug core; about 4%, w/w of the drug core; about 5%, w/w/ of the drug core, about 6%, w/w, of the drug core, about 7%, w/w, of the drug core, about 8%, w/w, of the drug core; about 9%, w/w, of the drug core; about 10% w/w of the drug core t; about 12%, w/w of the drug core; about 14%, w/w, of the drug core, about 16%, w/w/, of the drug core; about 18% w/w of the drug core; about 20%, w/w of the drug core, about 22%, w/w, of the drug core, about 24%, w/w, of the drug core; about 26% w/w of the drug core; about 28%, w/w of the drug core, about 30%, w/w, of the drug core, about 32%, w/w, of the drug core; between about 1% and about 10%, w/w of the drug core; between about 2% and about 8% w/w of the drug core; between about 3% and about 7% w/w of the drug core; between about 4% and about 6% w/w of the drug core.

Glidants can be added to improve the flow properties of a powder composition or granulate and improve the accuracy of dosing. Excipients that may function as glidants include colloidal silicon dioxide, magnesium trisilicate, powdered cellulose, starch, tribasic calcium phosphate, calcium silicate, powdered cellulose, colloidal silicon dioxide, magnesium silicate, magnesium trisilicate, silicon dioxide, starch, tribasic calcium phosphate, and talc. Appropriate amounts to use may be determined by those of skill in the art.

Permeation enhancers are an included excipient in one embodiment. Permeation enhancers act to enhance uptake of a substance through the intestinal wall and deliver more of a substance to the bloodstream. Movement through the intestinal wall may occur by passive diffusion, the movement of drug across a membrane in a manner driven solely by the concentration gradient; by carrier-mediated diffusion, movement of drug across a cell membrane via a specialized transport system embedded in the cell membrane; paracellular diffusion, the movement of drug across a membrane by going between, rather than through, two cells; and transcellular diffusion, the movement of a drug across the cell. Additionally, there are numerous cellular proteins capable of preventing intracellular accumulation of drugs by pumping drug that enters the cell back out. These are sometimes called efflux pumps. One of the most important is p-glycoprotein, which is present in many different tissues in the body (e.g., intestine, placental membrane, blood-brain barrier). Permeation enhancers can work by facilitating any of the processes mentioned above (such as by increasing fluidity of membranes, opening “tight junctions” between cells, and/or inhibiting efflux.)

Examples of suitable permeation inhibitors include, for example, but are not limited to, surfactants. Suitable examples for the present invention include are known and commercially available, e.g. from the BASF company under the trade mark SOLUTOL. An example is SOLUTOL HS15 which is known, e.g. from the BASF technical leaflet MEF 151E (1986), to comprise of about 70% polyethoxylated 12-hydroxystearate by weight and about 30% by weight unesterified polyethylene glycol component. SOLUTOL HS 15 has a hydrogenation value of 90 to 110, a saponification value of 53 to 63, an acid number of maximum 1, and a maximum water content of 0.5% by weight. Polyoxyethylene-polyoxypropylene co-polymers and block co-polymers are included in one embodiment, for example of the type known and commercially available under the trade names PLURONIC, EMKALYX and POLOXAMER. A further example of this class is POLOXAMER F127. Propylene glycol mono- and di-fatty acid esters such as propylene glycol dicaprylate (also known and commercially available under the trade name MIGLYOL 840), propylene glycol dilaurate, propylene glycol hydroxystearate, propylene glycol isostearate, propylene glycol laurate, propylene glycol ricinoleate, propylene glycol stearate and so forth are also included in some embodiments. Other examples include propylene glycol mono C:8 esters include SEFSOL 218 (Nikko Chemicals) and CAPRYOL 90 (Gattefosse) and tocopherol esters, e.g. tocopheryl acetate and tocopheryl acid succinate (HLB of about 16), transesterified ethoxylated vegetable oils are known and are commercially available under the trade name LABRAFIL. Examples are LABRAFIL M 2125 CS (obtained from corn oil and having an acid number of less than about 2, a saponification number of 155 to 175, an HLB value of 3 to 4, and an iodine number of 90 to 110), and LABRAFIL M 1944 CS (obtained from kernel oil and having an acid number of about 2, a saponification number of 145 to 175 and an iodine number of 60 to 90). LABRAFIL M 2130 CS (which is a transesterification product of a C.sub.12-18 glyceride and polyethylene glycol and which has a melting point of about 35 to 40.degree. C., an acid number of less than about 2, a saponification number of 185 to 200 and an iodine number of less than about 3). In one embodiment, the transesterified ethoxylated vegetable oil is LABRAFIL M 2125 CS which can be obtained, for example, from Gattefosse, Saint-Priest Cedex, France. In one embodiment, a permeation enhancer includes water soluble tocopheryl polyethylene glycol succinic acid esters (TPGS), e.g. with a polymerisation number ca 1000, e.g. available from Eastman Fine Chemicals Kingsport, Term., USA. Other embodiments include POLOXAMER compounds, particularly F127, chitosan, carboxymethylcellulose, SOLUTOL compounds, sodium laurate, and LABRAFIL compounds. Other permeation enhancers include alcohols, dimethyl sulfoxide, glyceryl monooleate, glycofurol, isopropyl myristate, isopropyl palmitate, lanolin, linoleic acid, myristic acid, oleic acid, oleyl alcohol, palmitic acid, polyoxyethylene alkyl ethers, 2-pyrrolidone, sodium lauryl sulfate, and thymol. Appropriate amounts to use can be determined by one of skill in the art.

In some embodiments, the present invention comprises a controlled release pharmaceutical composition for oral administration for enhanced systemic delivery of a cytidine analog comprising a therapeutically effective amount of a cytidine analog and a drug release controlling component which is capable of providing release of the cytidine analog primarily in the large intestine. The present invention in some embodiments includes a controlled release pharmaceutical composition for oral administration for enhanced systemic delivery of 5-azacytidine consisting essentially of or consisting of a therapeutically effective amount of 5-azacytidine and a drug release controlling component which is capable of providing release of the cytidine analog primarily in the large intestine. In other embodiments, the present invention includes a controlled release pharmaceutical composition for oral administration of a cytidine analog for enhanced systemic delivery of the cytidine analog consisting essentially or consisting of a therapeutically effective amount of a cytidine analog and an enteric coating which is capable of providing release of the cytidine analog primarily in the large intestine. In other embodiments, the present invention includes a controlled release pharmaceutical composition for oral administration for enhanced systemic delivery of a cytidine analog consisting essentially of (or consisting of) a therapeutically effective amount of 5-azacytidine and an enteric coating which is capable of providing release of the cytidine analog primarily in the large intestine and at least one excipient which improves the cohesive qualities of the, and/or increases the bulk of, and/or improves the manufacture of, or facilitates disintegration of, and/or retards the drug decomposition reactions occurring in, or enhances uptake through the intestinal wall of, the controlled release pharmaceutical compositions of the present invention.

A tablet can be made by compressing a powder composition granulate between a punch and dye. Some excipients and active ingredients have a tendency to adhere to the surfaces of the punch and dye, which can cause the tablet to have pitting and other surface irregularities. A lubricant may be added to the composition to reduce adhesion and ease release of the product form the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Suitable patients to treat include humans; birds such as chickens, ostriches, quail, and turkeys; mammals such as companion animals (including dogs, cats, and rodents) and economic food and/or fur or other product animals, such as horses, cattle, llamas, chinchillas, ferrets, goats, sheep, rodents, minks, rabbits, raccoons, and swine.

In another embodiment, the present invention includes a method for delivering a cytidine analog comprising administering to a patient in need thereof a composition of the present invention. In one embodiment, the composition comprises an oral formulation of a cytidine analog, wherein the oral formulation of the cytidine analog comprises a) a therapeutically effective amount of a cytidine analog and b) a drug release controlling component capable of providing release of the cytidine analog primarily in the large intestine, wherein after ingestion by a patient the cytidine analog is released primarily in the large intestine.

Another embodiment of the present invention includes a method of formulating a cytidine analog for oral delivery, comprising formulating (in one embodiment, coating) a therapeutically effective amount of a cytidine analog with a drug release controlling component capable of providing release of the cytidine analog primarily in the large intestine using methods disclosed in the present disclosure.

In another embodiment, the present invention includes a method of increasing the bioavailability of a cytidine analog comprising administering the controlled release pharmaceutical compositions of the present invention to a patient. Specifically, a controlled release pharmaceutical composition of the present invention is provided to a patient, and ingested by the patient, where the composition contacts the biological fluids of the patient's body and increases the bioavailability of the cytidine analog. Oral bioavailability of a cytidine analog in the compositions of the present invention can be more than 5%, more than 10%, more than 15%, more than 20%, more than 25%, more than 30% or more than 50% greater than the oral bioavailability of prior art formulations of a cytidine analog. Average maximum plasma concentration achieved relative to the dose administered may be more than 2 fold higher, 3 fold higher, 5 fold higher, about 10 fold higher than the oral bioavailability of prior art formulations of a cytidine analog when a cytidine analog is administered orally in the controlled release formulations of the present invention.

In another embodiment, the present invention includes methods for treating a patient having a disease associated with abnormal cell proliferation, comprising administering the controlled release pharmaceutical compositions of the present invention. In one embodiment, the controlled release pharmaceutical compositions of the present invention allow for enhanced bioavailability of the cytidine analog to the patient.

In some embodiments, indications that may be treated using the pharmaceutical compositions of the present invention include those involving undesirable or uncontrolled cell proliferations. Such indications include benign tumors, various types of cancers such as primary tumors and tumor metastasis, hematological disorders (e.g. leukemia, myelodysplastic syndrome and sickle cell anemia), restenosis (e.g. coronary, carotid, and cerebral lesions), abnormal stimulation of endothelial cells (arteriosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types of benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells that become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronmas, intestinal ganglioneuromas, hyperplastic corneal nerve tumor, marfanoid habitus tumor, Wilm's tumor, seminoma, ovarian tumor, leiomyoma tumor, cervical dysplasia and in situ carcinoma, neuroblastoma, retinoblastoma, medulloblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chormosomal breakage, such as Bloom's syndrome, Fanconi's anemia, Li-Fraumeni kindreds, ataxia-telangiectasia, and X-linked agammaglobulinemia.

Acute promyelocytic leukemia (APML) represents a distinct subgroup of AML. This subtype is characterized by promyelocytic blasts containing the 15; 17 chromosomal translocation. This translocation leads to the generation of the fusion transcript comprised of the retinoic acid receptor and a sequence PML.

Acute lymphoblastic leukemia (ALL) is a heterogeneous disease with distinct clinical features displayed by various subtypes. Reoccurring cytogenetic abnormalities have been demonstrated in ALL. The most common cytogenetic abnormality is the 9; 22 translocation. The resultant Philadelphia chromosome represents poor prognosis of the patient.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia. In one embodiment, MDS is a condition to treat with the present invention, and includes the following myelodysplastic syndrome subtypes: refractory anemia refractory anemia with ringed sideroblasts (if accompanied by neutropenia or thrombocytopenia or requiring transfusions), refractory anemia with excess blasts, refractory anemia with excess blasts in transformation, and chronic myelomonocytic leukemia.

Treatment of abnormal cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

Abnormal angiogenesis that may be may be treated using this invention include those abnormal angiogenesis accompanying rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanism of various angiogenic factors, including elements of the inflammatory response, such as leukocytes, platelets, cytokines, and eicosanoids, or unidentified plasma constituents have yet to be revealed. The pharmaceutical composition of the present invention may also be used for treating diseases associated with undesired or abnormal angiogenesis alone or in conjunction with an anti-angiogenesis agent.

The particular dosage of these agents required to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases. According to this embodiment, the pharmaceutical composition of the present invention may be used to treat a variety of diseases associated with undesirable angiogenesis such as retinal/choroidal neuvascularization and corneal neovascularization. Examples of retinal/choroidal neuvascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disease, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

The pharmaceutical composition of the present invention may be used for treating chronic inflammatory diseases associated with abnormal angiogenesis. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis using the composition of the present invention may prevent the formation of the granulomas, thereby alleviating the disease. Examples of chronic inflammatory disease include, but are not limited to, inflammatory bowel diseases such as Crohn's disease and ulcerative colitis, psoriasis, sarcoidosis, and rheumatoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea. These inflammatory bowel diseases are generally caused by chronic granulomatous inflammation throughout the gastrointestinal tract, involving new capillary sprouts surrounded by a cylinder of inflammatory cells. Inhibition of angiogenesis by the composition of the present invention should inhibit the formation of the sprouts and prevent the formation of granulomas. The inflammatory bowel diseases also exhibit extra intestinal manifestations, such as skin lesions. Such lesions are characterized by inflammation and angiogenesis and can occur at many sites other the gastrointestinal tract. Inhibition of angiogenesis by the composition of the present invention should reduce the influx of inflammatory cells and prevent the lesion formation.

Sarcoidois, another chronic inflammatory disease, is characterized as a multisystem granulomatous disorder. The granulomas of this disease can form anywhere in the body and, thus, the symptoms depend on the site of the granulomas and whether the disease is active. The granulomas are created by the angiogenic capillary sprouts providing a constant supply of inflammatory cells. By using the composition of the present invention to inhibit angionesis, such granulomas formation can be inhibited. Psoriasis, also a chronic and recurrent inflammatory disease, is characterized by papules and plaques of various sizes. Treatment using the composition of the present invention should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the composition of the present invention alone or in conjunction with other anti-RA agents should prevent the formation of new blood vessels necessary to maintain the chronic inflammation and provide the RA patient relief from the symptoms.

The pharmaceutical composition of the present invention may also be used to treat autoimmune diseases. Autoimmune diseases refer to a wide range of degenerative diseases caused by the immune system attacking a person's own cells. Autoimmune diseases are usually classified clinically in a variety of ways. In light of affected parts by the diseases, there are, for example, degenerative diseases of supporting tissues and connective tissues; autoimmune degenerative diseases of salivary glands, particularly Sjogren's disease; autoimmune degenerative diseases of kidneys, particularly systemic lupus erythematodes (SLE) and glomerulonephritis; autoimmune degenerative diseases of joints, particularly rheumatoid arthritis; and autoimmune degenerative diseases of blood vessels such as generalized necrotizing angitis and granulomatous angitis; and multiple sclerosis. Alternatively, autoimmune diseases can be classified in one of the two different categories: cell-mediated disease (i.e. T-cell) or antibody mediated disorders. Examples of cell-mediated autoimmune diseases include multiple sclerosis, rheumatoid arthritis, autoimmune thyroiditis, and diabetes mellitus. Antibody-mediated autoimmune disorders include myasthenia gravis and SLE.

Dosing schedules for the compositions and methods of the present invention, for example, can be adjusted to account for the patient's characteristics and disease status. Appropriate dose will depend on the disease state being treated. Appropriate biomarkers may be used to evaluate the drug's effects on the disease state and provide guidance to the dosing schedule. In some cases, daily doses, and in others, selected days of a week, month or other time interval. In one embodiment, the drug will not be given more than once per day. In one embodiment, dosing schedules for administration of pharmaceutical compositions of the present invention including the daily administration to a patient in need thereof of. Dosing schedules may mimic those that are used for non-oral formulations of a cytidine analog, adjusted to maintain, for example, substantially equivalent therapeutic concentration in the patient's body.

The following examples are provided for illustrative purposes only and are not intended to limit the scope of the invention.

### EXAMPLES

### Example 1

Absorption Potential Assessment of 5-azacytidine Using Caco 2 Monolayers

The permeability of 5-azacytidine was determined in a Caco 2 monolayer model system using phosphate buffered saline as the system medium. The Caco-2 cells are an intestinal epithelial cell line (human colon adenocarcinoma established from the primary colon tumor (adenocarcinoma)) Monolayers of Caco-2 cells are used to classify the intestinal absorption potential of a drug candidate molecule. The assay was carried out in accordance with P. Artursson and J. Karlsson, “Correlation between Oral Drug Absorption in Humans and Apparent Drug Permeability Coefficients in Human Intestinal Epithelial (Caco-2) Cells”, Biochem. Biophys. Res. Commun. 175, 880 (1991).

Briefly, Caco-2 cells were grown to confluence on collagen-coated, microporous polycarbonate in 12-well plates. For the assay buffer, Dulbecco's Phosphate Buffered Saline at pH 7.4 was used. The chamber on the apical side of the cells was filled with DPBS containing 1000 micromolar 5-azacytidine with or without additional excipients. The test compound was then dosed on either the apical or basolateral side of the Caco-2 monolayer, and flux across the monolayer was determined both one and two hours after dosing. Results were compared to control high permeability compounds metoprolol and antipyrine and low permeability compounds atenolol and ranitidine, and were expressed as Papp (apparent permeability) compared to reference compound standards. Integrity of the monolayers was determine both before and after testing by measuring the transendothelial electrical resistance (TEER) value. Apparent permeability is calculated as (dCᵣ/dt)×Vᵣ/(A×C₀) where dCᵣ/dt is the slope of the cumulative concentration in the receiver compartment versus time in micromolar per second, Vᵣ is the volume of the receiver compartment in cubic centimeters, A is the area of the cell monolayer, and C₀ is the measured initial donor concentration in micromolar.

In the absence of additional excipients the measured apical to basolateral 5-azacytidine permeability for 5-azacytidine was 0.15±0.02×10⁻⁶ cm/sec.

Additionally, a series of pharmaceutically acceptable excipients were screened for their ability to increase the apparent permeability of 5-azacytidine in this model system. The excipients evaluated and their effects on 5-azacytidine permeability in this model system are presented in Table 1.

Table 1. Sodium laurate was obtained from Sigma Chemical (available from St. Louis, Mo.); Vitamin E TPGS (TPGS-TPGS-d-alpha tocopherol polyethylene glycol 1000 succinate (available from Eastman, Kingsport, Tenn.); LABRAFIL M 1944 CS (2) (Oleyl macrogolglycerides) (available from Gattefosse, France).

TABLE 1
Effect of 5-azacytidine Permeability in Caco 2 Monolayer Model
with Various Excipients

| | Excipient | |
|---|---|---|
| Excipient | Concentration (%) | Pₐₚₚ (×10⁻⁶ cm/sec) |
| Control | NA | 0.15 ± 0.02 |
| Poloxamer F127 | 0.1 | 0.13 ± 0.02 |
| Chitosan | 0.1 | 0.15 ± 0.01 |
| Carboxymethyl Cellulose | 0.1 | 0.26 ± 0.09 |
| Solutol | 0.1 | 0.26 ± 0.04 |
| Sodium Laurate | 0.1 | 15.77 ± 1.77* |
| | 0.05 | 0.17 ± 0.03 |
| | 0.01 | 0.19 ± 0.02 |
| | 0.001 | 0.19 ± 0.04 |
| Labrafil | 0.1 | 0.34 ± 0.002 |
| | 0.05 | 0.38 ± 0.13 |
| | 0.01 | 0.23 ± 0.04 |
| | 0.001 | 0.27 ± 0.02 |
| TPGS | 0.1 | 0.38 ± 0.09 |
| | 0.05 | 0.45 ± 0.18 |
| | 0.01 | 0.30 ± 0.05 |
| | 0.001 | 0.33 ± 0.08 |

*High permeability due to toxic effects of sodium laurate on Caco 2 cells

### CONCLUSION

5-azacytidine was found to permeate through Caco-2 monolayers, indicating that colonic epithelial cells are a good candidate for delivery of 5-azacytidine for enhanced bioavailability. Permeation of 5-azacytidine was found to be enhanced by TPGS and Labrafil, having a significant effect with respect to increased 5-azacytidine permeability. For both excipients, there appeared to be a shallow dose response relationship between the amount of excipient and the observed permeability. Inclusion of appropriate amounts of TPGS and/or Labrafil results in an increase in the apparent permeability of 5-azacytidine without adverse effects on Caco 2 monolayers. Inclusion of one or both of these excipients should improve oral bioavailability of 5-azacytidine through enhanced GI absorption.

### Example 2

Permeability in Human Intestinal Strips

The permeability of 5-azacytidine has also been assessed in viable human intestinal strips derived from specific sections of the GI tract. This model allows evaluation of the absorption potential of drugs and differences in drug absorption across the human jejunum, ileum, and colon and is based on Ungell et al. “Membrane Transport of Drugs in Different Regions of the Intestinal Tract of the Rat”, J. Pharm. Sci. 87:360-366, (1998) and Nejdfors et al. “Mucosal in vitro Permeability in the Intestinal Tract of the Pig, the Rat, and Man: Species and Region Related Differences”, Scand. J. Gastroenterol. 35:501-507, (2000). Permeation experiments were performed for 5-azacytidine in the jejunal, ileal, and colonic tissues originating from the same human donor. Post-mortem human whole intestine was obtained from the International Institute for the Advancement of Medicine (IIAM). Tissues were used within 24 hours of the organ's removal. Tissues were maintained in cold transport media before being stripped. The segments were cut along the mesenteric border, the underlying musculature was stripped off, and the epithelium was rinsed with ice cold normal saline. The stripped tissues were mounted within the vertical USSING chambers (Harvard Apparatus, Holliston, Mass.). In these studies, 5-azacytidine permeability was measured in both absolute rate and relative to atenolol (internal control). In all cases the net apparent permeability was assessed in the mucosal to serosal direction only. Evaluation conditions included the use of 5-azacytidine alone and for some samples 5-azacytidine in the presence of ketoconazole and THU as enzymatic inhibitors of CYP3A4 and cytidine deaminase, respectively.

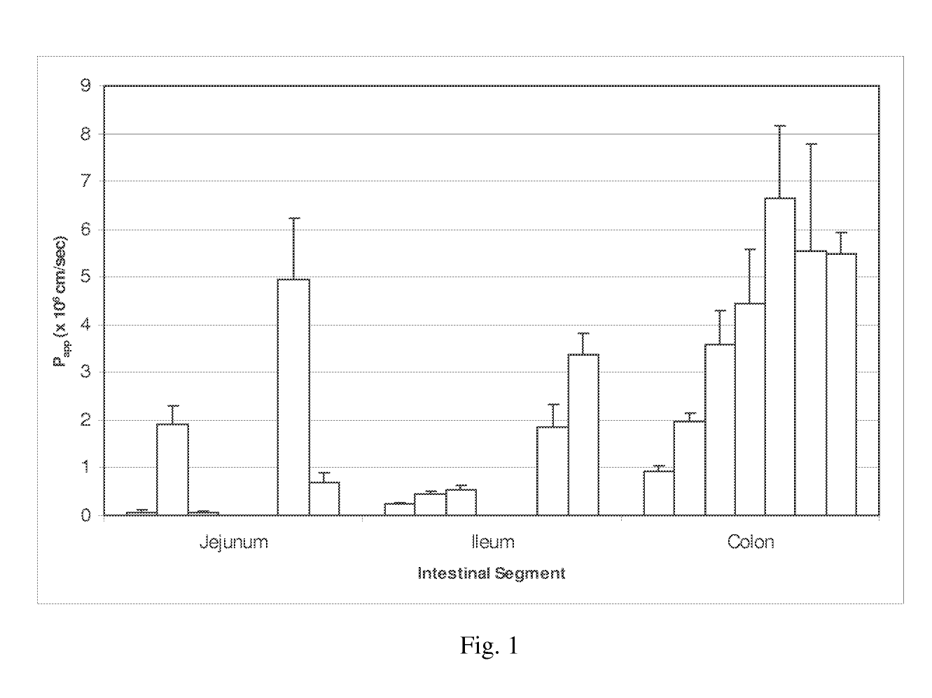

Absolute permeability values for multiple donors at different intestinal sites with and without enzymatic inhibition are presented in graphical form in FIG. 1. FIG. 1 shows absolute mucosal to serosal permeability of 5-azacytidine in human intestinal tissue with and without enzymatic inhibition. In general permeability appeared greatest in the colon relative to the jejunum and ileum. Inclusion of enzymatic inhibitors increased the absolute permeability in all GI tract regions, however the size of the increase was maximal in the jejunum and ileum and was relatively small in the colon.

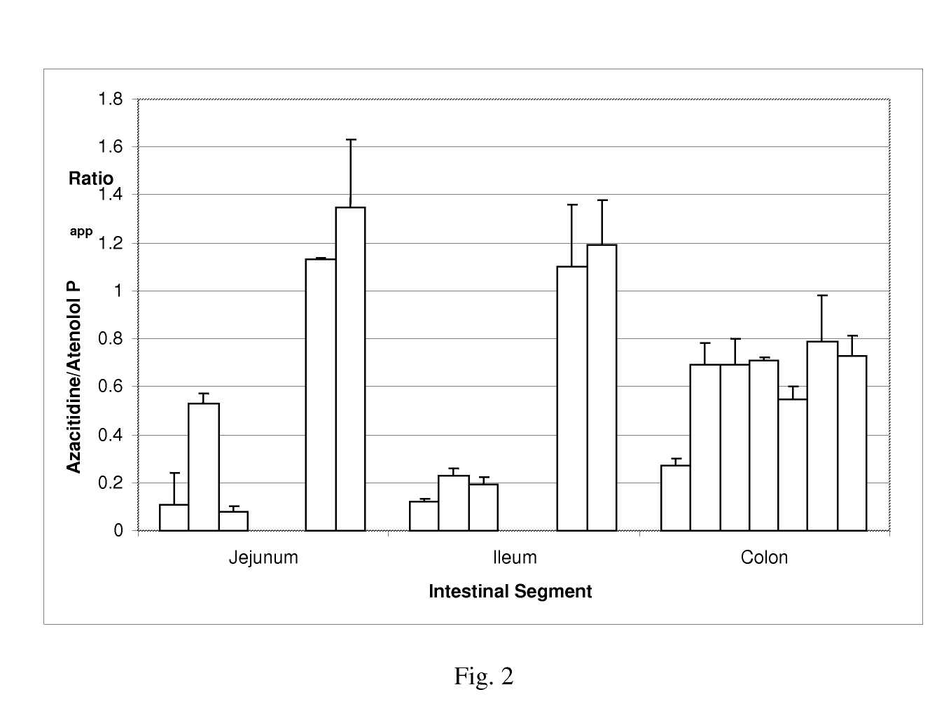

The permeability of 5-azacytidine in the same human intestinal strips relative to the internal control (atenolol) is shown graphically in FIG. 2. FIG. 2 shows relative mucosal to serosal permeability of 5-azacytidine in human intestinal tissue with and without enzymatic inhibition with respect to atenolol. The use of internal controls ensures that small variations due to tissue viability and processing are normalized. Qualitatively, the same conclusions can be drawn from the 5-azacytidine permeability data relative to atenolol. Absorption was greatest and most consistent between donors in colonic tissue, and the effects of enzymatic inhibition were minimized in tissues derived from the colon.

Permeation Enhancement in Human Tissues

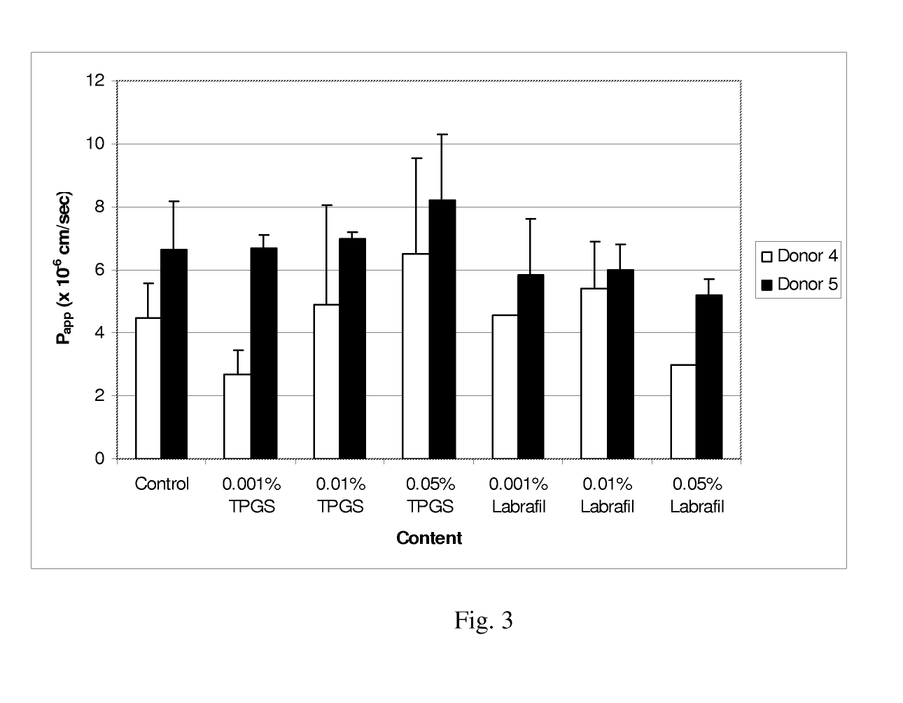

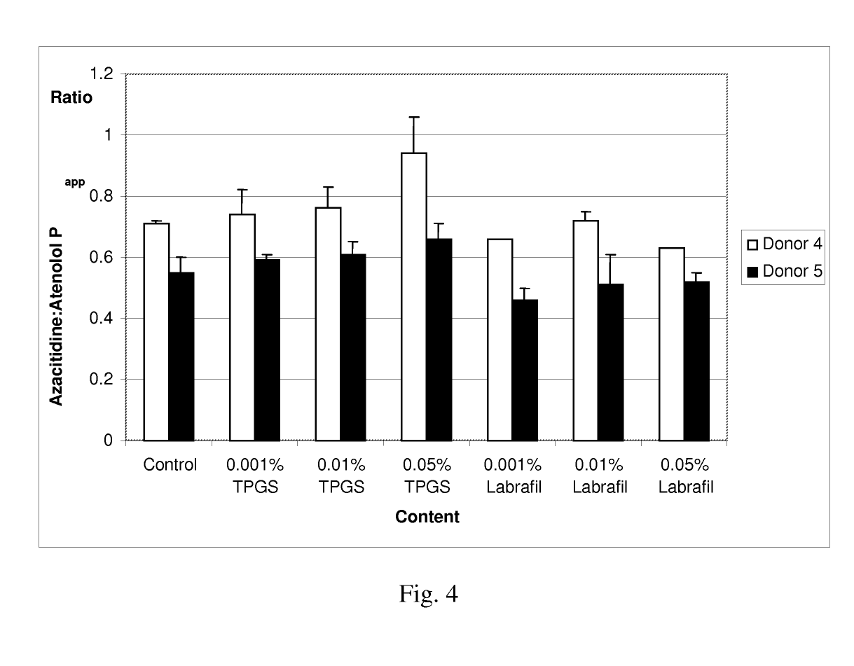

Evaluation of the effects of 5-azacytidine permeability in human colonic strips with various levels of the permeation enhancers identified in the Caco 2 model system has also been performed. Absolute and relative permeability of 5-azacytidine with the two excipients at various levels are presented in FIGS. 3 and 4, respectively. FIG. 3 shows absolute mucosal to serosal permeability of 5-azacytidine in human colonic tissue with various concentrations of TPGS or LABRAFIL without enzymatic inhibition, and FIG. 4 shows relative mucosal to serosal permeability of 5-azacytidine in human colonic tissue with various concentrations of TPGS or LABRAFIL without enzymatic inhibition.

There was a trend toward greater permeability with increasing levels of TPGS, albeit to a lesser extent than observed in the model Caco2 system. Conversely, under the same conditions Labrafil did not appear to affect 5-azacytidine permeability in human colonic tissue.

### CONCLUSIONS

The available data from viable human intestinal tissue shows that enzymatic degradation of 5-azacytidine was more prevalent in upper GI segments. Effective administration of 5-azacytidine with delivery to the jejunum and/or ileum may require the use of an enzymatic inhibitor such as THU. By contrast, both the absolute and relative permeability of 5-azacytidine appeared maximal in human colonic tissue. Furthermore, enzymatic inhibition in colonic tissue did not provide a dramatic improvement in 5-azacytidine permeability. Therefore, an oral 5-azacytidine dosage form that targets the colon for delivery has been shown to maximize bioavailability without the need to include an enzymatic inhibitor.

### Example 3

Solid Oral Dosage Form

Solid oral dosage forms of 5-azacytidine were prepared using standard pharmaceutical excipients and techniques. TPGS was first adsorbed onto either microcrystalline cellulose or calcium silicate in an independent step. Dry ingredients were then dry blended and tablets prepared by direct compression. Tablets were then enteric coated with EUDRAGIT S100 from an acetone—isopropanol solvent mixture or with AQUAT AS-HG from a methylene chloride—ethanol solvent mixture.

Clinical Trial Material cores were composed of the following materials in these ratios:

| Ingredient | mg/tablet | % w/w |
|---|---|---|
| 5-azacytidine | 20.0 | 20.0 |
| Mannitol, USP | 58.2 | 58.2 |
| Microcrystalline Cellulose, NF | 15.0 | 15.0 |
| Crospovidone, NF | 3.0 | 3.0 |
| Magnesium Stearate, NF | 1.8 | 1.8 |
| Vitamin E TPGS, NF | 2.0 | 2.0 |

Cores were then be coated to approximately 7% w/w with the following mixture:

| Ingredient | mg/tablet | % w/w |
|---|---|---|
| EUDRAGIT S100, NF | 5.0 | 71.5 |
| Triethyl citrate, NF | 0.5 | 7.0 |
| Talc, USP | 1.5 | 21.5 |

Excipient compatibility studies have demonstrated that 5-azacytidine is compatible with each excipient. Stability studies have demonstrated excellent stability of both cores and coated tablets under long term (25° C., 60% RH) and accelerated (40°, 70% relative humidity) storage conditions. The formulation composition that has been manufactured is presented in Table 2.

TABLE 2
Oral 5-azacytidine Tablet Composition

| | | | Quality |
|---|---|---|---|
| Material | Trade Name | Purpose | Standard |
| 5-azacytidine | NA | Active | In-House |
| Mannitol | PARTECK | Bulking Agent | USP |
| | M200 | | |
| Microcrystalline | PROSOLV | Binding Agent | USP |
| Cellulose | 90HD | | |
| Crospovidone | POLYPLASONE | Disintegrant | USP |
| | XL | | |
| Magnesium Stearate | NA | Lubricant | USP |
| Vitamin E TPGS | NA | Absorption | NF |
| | | Enhancement | |
| Methacrylic acid | EUDRAGIT | Enteric Coating | USP |
| copolymer¹ | S100² | | |
| Triethyl Citrate | MORFLEX | Plasticizer | USP |
| Talc, | | Anticaking Agent | USP |

¹Hypromellose Acetate Succinate, NF alternate material
²AQUAT AS-HG, alternate trade name

### Example 4

Solid Oral Dosage Form

Solid oral dosage forms of 5-azacytidine were prepared using standard pharmaceutical excipients and techniques. TPGS was first adsorbed onto either microcrystalline cellulose or calcium silicate in an independent step. Dry ingredients were then dry blended and tablets prepared by direct compression. Tablets were then film coated with Klucel EF from ethanol followed by enteric coated with EUDRAGIT S1100 from an acetone—isopropanol solvent mixture.

Clinical Trial Material cores were composed of the following materials in these ratios:

| Ingredient | mg/tablet | % w/w |
|---|---|---|
| 5-azacytidine | 20.0 | 20.0 |
| Mannitol, USP | 43.2 | 43.2 |
| Microcrystalline Cellulose, NF | 30.0 | 30.0 |
| Crospovidone, NF | 3.0 | 3.0 |
| Magnesium Stearate, NF | 1.8 | 1.8 |
| Vitamin E TPGS, NF | 2.0 | 2.0 |

Cores were then sub-coated to approximately 4% w/w with the following materials in ethanol:

| Ingredient | mg/tablet | % w/w |
|---|---|---|
| Klucel EF, NF | 4.0 | 6.0 |

Film coated cores were then be coated to approximately 7% w/w with the following mixture:

| Ingredient | mg/tablet | % w/w |
|---|---|---|
| Eudragit S100, NF | 6.0 | 86.3 |
| Triethyl citrate, NF | 1.0 | 13.7 |

Excipient compatibility studies have demonstrated that 5-azacytidine is compatible with each excipient. Stability studies have demonstrated excellent stability of coated tablets under long term (25° C., 60% RH) and accelerated (40°, 70% RH) storage conditions. An additional formulation composition that has been manufactured is presented in Table 3.

TABLE 3
Oral 5-azacytidine Tablet Composition

| | | | Quality |
|---|---|---|---|
| Material | Trade Name | Purpose | Standard |
| 5-azacytidine | NA | Active | In-House |
| Mannitol | Parteck M200 | Bulking Agent | USP |
| Microcrystalline | Prosolv 90HD | Binding Agent | USP |
| Cellulose | | | |
| Crospovidone | Polyplasone XL | Disintegrant | USP |
| Magnesium Stearate | NA | Lubricant | USP |
| Vitamin E TPGS | NA | Absorption | NF |
| | | Enhancement | |
| Hydroxypropyl Cellulose | Klucel EF | Sub-Coating | NF |
| Methacrylic acid | Eudragit S100² | Enteric Coating | USP |
| copolymer¹ | | | |
| Triethyl Citrate | Morflex | Plasticizer | USP |

¹Hypromellose Acetate Succinate, NF alternate material
²Aquat AS-HG, alternate trade name

### Example 5

A clinical study using the oral formulation described in Example 3 was performed to assess the safety and bioavailability of single oral doses of 5-azacytidine in patients with myelodysplastic syndromes, acute myelogenous leukemia, or solid tumors. The study was a multicenter, open-label, single treatment study. Patients were treated with escalating doses, in 20 mg increments, up to 200 mg. The study assessed the safety and tolerability of escalating doses, provided pilot information on the oral bioavailability of the study drug, and provided information on the single dose pharmacokinetics of the study drug after oral administration.

Study Design

Multicenter, open-label, single-treatment, escalating-dose PK study. 1 subject was to receive an oral dose of 5-azacytidine 60 mg (three 20 mg tablets). Single subject cohorts were used for each dose escalation. Subsequent subjects were to be treated at escalating doses, up to 200 mg, in 20 mg increments. Dose escalation was to continue until 1 of the following conditions was reached:
a. Drug was deemed intolerable (i.e., if a subject experienced any Grade 3 or 4 adverse event [AE] possibly related to 5-azacytidine, or the investigator identified any safety concern following drug treatment); or
b. Appropriate concentrations were achieved (defined as >4 consecutive timed plasma samples containing quantifiable 5-azacytidine concentrations amenable to PK assessments); or
c. Dose escalation reached the 200 mg level, which is approximately equivalent to the maximum approved daily SC dose of 5-azacytidine (i.e., 100 mg/m²).

If drug was deemed intolerable, a 2^{nd}, and then a 3^{rd}, subject were to be treated at the same dose to confirm intolerance. If no safety concerns were identified and no Grade 3 or 4 AEs occurred in these subjects, dose escalation was to continue. When appropriate concentrations of drug were achieved, 1-2 additional subjects were treated at the same dose to verify results. After an overnight (8-hour) fast, each subject received a single oral dose of 5-azacytidine. Serial blood samples for plasma PK analyses were drawn before, and at the following time points after, dosing: 0.5, 1, 1.5, 2, 2.5, 3, 3.5, 4, 4.5, 5, 5.5, 6, 6.5, 7, 8 hours, and and 12 hours (if possible). A validated high-performance liquid chromatography/tandem mass spectrometric method (LC-MS/MS) was used to determine 5-azacytidine concentrations in plasma. PK parameters calculated from plasma concentrations included (but were not limited to) Cₘₐₓ, Tₘₐₓ, t_{1/2}, and AUC_{(0-∞)}.

Patients

Inclusion Criteria:
a. Male or female subjects with MDS, AML, or malignant solid tumors, ≧18 years of age, with Eastern Cooperative Oncology Group (ECOG) performance status 0-2 were eligible.
b. For subjects with AML or malignant solid tumors, eligibility was limited to those for whom standard curative or palliative measures did not exist or were no longer effective.
c. Patients must have had normal renal, hepatic, and gastrointestinal function.

Exclusion Criteria:
a. Pregnancy
b. History of severe cardiac or pulmonary disease
c. Advanced malignant hepatic tumor
d. Receipt of radiation therapy, chemotherapy, or investigational drugs within 30 days of planned testing.

Patients (Demographic and Disease Characteristics are Shown in Table 4.)

4 subjects were enrolled and received study drug
a. 1 subject received a 60 mg dose which was well tolerated. 5-azacytidine was quantifiable in plasma in 2 samples and the dose was escalated.
b. 1 subject received an 80 mg dose which was well tolerated. 5-azacytidine was detectable in 4 consecutive samples. Two additional subjects were then treated at the 80 mg level.

TABLE 4

| Subject | | | Date of | | | Oral Aza |
|---|---|---|---|---|---|---|
| (pt. #) | Age | Sex | Diagnosis | Tumor Type | ECOG Status | Dose |
| 1 (101) | 43 | M | May 1990 | Metastatic thymic carcinoid, | 1 (restricted) | 60 mg |
| | | | | mets to lung and skin lesions | | |
| 2 (102) | 67 | M | December 2000 | Prostate cancer | 0 (fully active) | 80 mg |
| 3 (201) | 57 | M | December 2006 | AML | 2 (ambulatory, | 80 mg |
| | | | | | capable of self care) | |
| 4 (202) | 65 | M | November 2006 | MDS secondary to successfully | 1 (restricted) | 80 mg |
| | | | | treated AML (CR achieved) | | |

PK Results

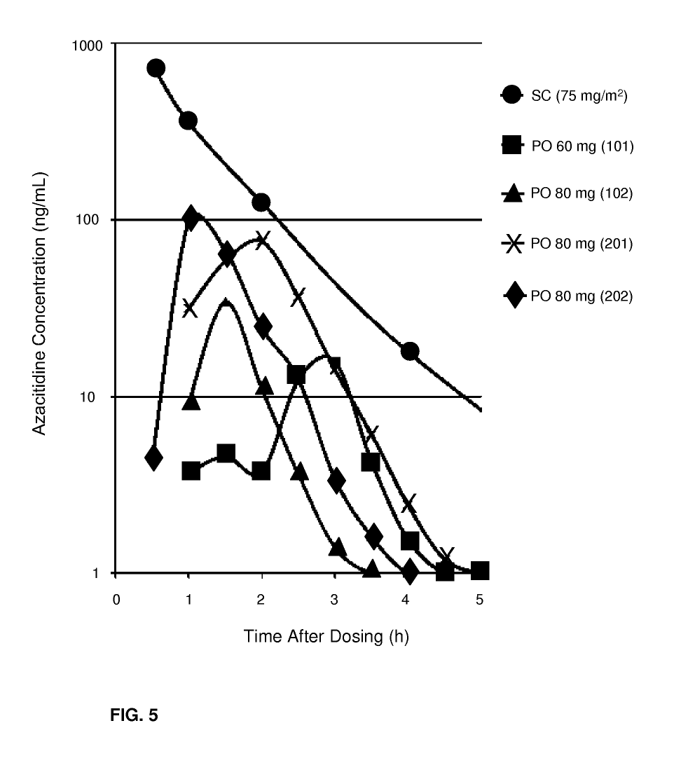

Concentration vs time profiles of individual subjects are shown in FIG. 5 (semi-logarithmic scale)
a. Cₘₐₓ for subjects who received an 80 mg dose were approximately 2-, 5-, and 6-fold higher than that of the subject dosed at 60 mg.
b. Bioavailability of the 80 mg oral dose relative to SC dosing was 6.3%, 24%, and 22%.
c. Tₘₐₓ for subjects who received an 80 mg dose occurred at 1.5, 2.0, and 1.0 h post-dose.

5-azacytidine plasma concentrations for each subject, the mean concentration of the 80 mg dose group (n=3), and mean SC 5-azacytidine concentrations (historical data; n=6) are presented in Table 5.

TABLE 5
5-azacytidine Plasma Concentrations (ng/mL)

| Oral Azacitidine | | | | | | SC Azacitidine | |
|---|---|---|---|---|---|---|---|
| Concentrations (ng/mL) | | | | | | Concentrations | |
| | Subject # | | | | | (ng/mL) | |
| Time | 101 | 102 | 201 | 202 | Mean | Time | 75 mg/m² |
| (h) | (60 mg) | (80 mg) | (80 mg) | (80 mg) | (80 mg) | (h) | SC |
| 0 | 0 | 0 | 0 | 0 | 0 | 0 | 0 |
| 0.5 | BLQ | BLQ | BLQ | 4.46 | 1.49 | 0.5 | 750 |
| 1.0 | 3.72 | 9.25 | 31.5 | 99.1 | 46.6 | 1 | 354.2 |
| 1.5 | 4.56 | 34.3 | 58.5 | 66.7 | 53.2 | 2 | 124.5 |
| 2.0 | 3.75 | 11 | 75.1 | 24.5 | 36.9 | 4 | 17.9 |
| 2.5 | 12.4 | 3.61 | 36.4 | 12.5 | 17.5 | 8 | BLQ |
| 3.0 | 15.8 | 1.4 | 14.1 | 3.41 | 6.30 | | |
| 3.5 | 4.27 | BLQ | 6.1 | 1.61 | 2.57 | | |
| 4.0 | 1.55 | BLQ | 2.43 | BLQ | BLQ | | |
| 4.5 | BLQ | BLQ | 1.19 | BLQ | BLQ | | |
| 5.0 | BLQ | BLQ | BLQ | BLQ | BLQ | | |

BLQ = below limit of quantitation

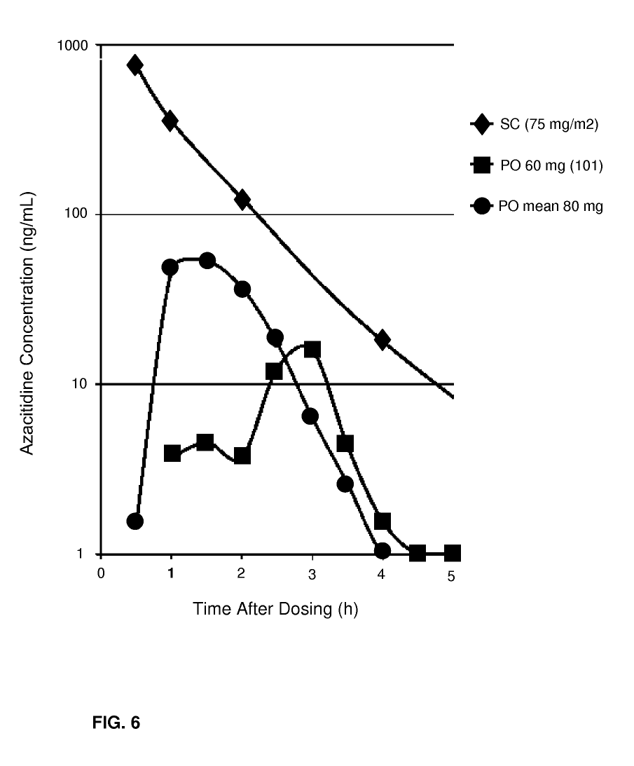

Concentration vs time profiles for the 60 mg dose and the mean of the three 80 mg doses are shown in FIG. 6 (semi-logarithmic scale).

For the mean 80 mg dose:
a. Concentrations were below the level of quantitation at 4.0 h
b. Tₘₐₓ was 1.5 h
c. Cₘₐₓ increased 3.5-fold versus the 60 mg dose
d. Bioavailability relative to the historical SC 5-azacytidine group was 18%.

A summary of results of PK measurements is presented in Table 6.

TABLE 6

| | | AUC_{(0-∞)} | | | | |
|---|---|---|---|---|---|---|
| | | (ng · | Cₘₐₓ | | | |
| Subject # | Dose | h/mL) | (ng/mL) | t_{1/2} (h) | Tₘₐₓ (h) | F (%)* |
| 101 | 60 mg | 22.6 | 15.8 | 0.299 | 3.0 | 6.7 |
| 102 | 80 mg | 28.6 | 34.3 | 0.336 | 1.5 | 6.3 |
| 201 | 80 mg | 111 | 75.1 | 0.416 | 2.0 | 24 |
| 202 | 80 mg | 103 | 99.1 | 0.366 | 1.0 | 22 |
| Mean (n = 3) | 80 mg | 81.5 | 53.2 | 0.361 | 1.5 | 18 |

*Percent bioavailability compared with historical SC azacitidine data (dose = 135 mg, AUC = 777 ng · h/mL)

Study endpoint was reached following evaluation of 4 subjects.

Safety

All AEs possibly related to study drug were Grade 2. No study-drug-related SAEs were reported.

The outcome from the clinical trial was shown to be that patients dosed with the referenced oral 5-azacytidine formulation showed measurable levels of 5-azacytidine in plasma samples. The amount of 5-azacytidine measured in the plasma was proportional to the administered dose, and the apparent oral bioavailability is acceptable for therapeutic treatment. The conclusions drawn from such results are that the current formulation delivered 5-azacytidine to the colonic region, and that site was shown to be capable of efficiently absorbing 5-azacytidine without the degradation associated with cytidine deaminase.

### Example 6

Solid Oral Dosage Form

Solid oral dosage forms of 5-azacytidine were prepared using standard pharmaceutical excipients and techniques. TPGS was first adsorbed onto either microcrystalline cellulose or calcium silicate in an independent step. Dry ingredients were then dry blended and tablets prepared by direct compression. Tablets were then film coated with Klucel EF from ethanol followed by film coating with a mixture of Eudragit RS PO, triethyl citrate, pectin and chitosan from an ethanol—acetone mixture. Eudragit RS PO is a water insoluble copolymer of methacrylic acid and aminoethylmethacrylic acid which has low permeability and pH independent swelling characteristics. Its inclusion in the film coat is to facilitate film formation rather than provide a pH dependent barrier to dissolution Pectin—chitosan complex was prepared via neutralization of an acidic 1:1 mixture of aqueous pectin and chitosan followed by collection and drying of the prepared solid.

Clinical Trial Material cores were composed of the following materials in these ratios:

| Ingredient | mg/tablet | % w/w |
|---|---|---|
| 5-azacytidine | 20.0 | 20.0 |
| Mannitol, USP | 43.2 | 43.2 |
| Microcrystalline Cellulose, NF | 30.0 | 30.0 |
| Crospovidone, NF | 3.0 | 3.0 |
| Magnesium Stearate, NF | 1.8 | 1.8 |
| Vitamin E TPGS, NF | 2.0 | 2.0 |

Cores were then sub-coated to approximately 4% w/w with the following materials in ethanol:

| Ingredient | mg/tablet | % w/w |
|---|---|---|
| Klucel EF, NF | 4.0 | 6.0 |

Film coated cores were then be coated to approximately 9% w/w with the following mixture:

| Ingredient | mg/tablet | % w/w |
|---|---|---|
| Eudragit RS PO, NF | 5.0 | 55.6 |
| Triethyl citrate, NF | 1.0 | 11.2 |
| Pectin, USP | 1.5 | 16.6 |
| Chitosan, Low MW | 1.5 | 16.6 |

Excipient compatibility studies have demonstrated that 5-azacytidine is compatible with each excipient. Stability studies have demonstrated excellent stability of coated tablets under long term (25° C., 60% RH) and accelerated (40°, 70% RH) storage conditions. An additional formulation composition that has been manufactured is presented in Table 7.

TABLE 7
Oral 5-azacytidine Tablet Composition

| | | | Quality |
|---|---|---|---|
| Material | Trade Name | Purpose | Standard |
| 5-azacytidine | NA | Active | In-House |
| Mannitol | Parteck M200 | Bulking Agent | USP |
| Microcrystalline | Prosolv 90HD | Binding Agent | USP |
| Cellulose | | | |
| Crospovidone | Polyplasone XL | Disintegrant | USP |
| Magnesium Stearate | NA | Lubricant | USP |
| Vitamin E TPGS | NA | Absorption | NF |
| | | Enhancement | |
| Hydroxypropyl | Klucel EF | Sub-Coating | NF |
| Cellulose | | | |
| Methacrylic acid | Eudragit RS PO | Film Coating | USP |
| copolymer | | | |
| Triethyl Citrate | Morflex | Plasticizer | USP |
| Pectin | CP Kelco | Colonic Selective | USP |
| | Kelcogel | Releasing Agent | |
| Chitosan, Low MW | NA | Colonic Selective | In-House |
| | | Releasing Agent | |

### Example 7

Solid Oral Dosage Form

Solid oral dosage forms of 5-azacytidine were prepared using standard pharmaceutical excipients and techniques. TPGS was first adsorbed onto either microcrystalline cellulose or calcium silicate in an independent step. Dry ingredients were then dry blended and tablets prepared by direct compression. Tablets were then film coated with Klucel EF from ethanol followed by film coating with a mixture of Eudragit RS PO, triethyl citrate, and amylose acetate from an ethanol—diethyl ether mixture. Eudragit RS PO is a water insoluble copolymer of methacrylic acid and aminoethylmethacrylic acid which has low permeability and pH independent swelling characteristics. Its inclusion in the film coat is to facilitate film formation rather than provide a pH dependent barrier to dissolution.

Clinical Trial Material cores were composed of the following materials in these ratios:

| Ingredient | mg/tablet | % w/w |
|---|---|---|
| 5-azacytidine | 20.0 | 20.0 |
| Mannitol, USP | 43.2 | 43.2 |
| Microcrystalline Cellulose, NF | 30.0 | 30.0 |
| Crospovidone, NF | 3.0 | 3.0 |
| Magnesium Stearate, NF | 1.8 | 1.8 |
| Vitamin E TPGS, NF | 2.0 | 2.0 |

Cores were then sub-coated to approximately 4% w/w with the following materials in ethanol:

| Ingredient | mg/tablet | % w/w |
|---|---|---|
| Klucel EF, NF | 4.0 | 6.0 |

Film coated cores were then be coated to approximately 9% w/w with the following mixture:

| Ingredient | mg/tablet | % w/w |
|---|---|---|
| Eudragit RS PO, NF | 4.0 | 44.5 |
| Triethyl citrate, NF | 0.75 | 8.3 |
| Amylose acetate | 4.25 | 47.2 |

Excipient compatibility studies have demonstrated that 5-azacytidine is compatible with each excipient. Stability studies have demonstrated excellent stability of coated tablets under long term (25° C., 60% RH) and accelerated (40°, 70% RH) storage conditions. An additional formulation composition that has been manufactured is presented in Table 8.

TABLE 8
Oral 5-azacytidine Tablet Composition

| | | | Quality |
|---|---|---|---|
| Material | Trade Name | Purpose | Standard |
| 5-azacytidine | NA | Active | In-House |
| Mannitol | Parteck M200 | Bulking Agent | USP |
| Microcrystalline | Prosolv 90HD | Binding Agent | USP |
| Cellulose | | | |
| Crospovidone | Polyplasone XL | Disintegrant | USP |
| Magnesium Stearate | NA | Lubricant | USP |
| Vitamin E TPGS | NA | Absorption | NF |
| | | Enhancement | |
| Hydroxypropyl | Klucel EF | Sub-Coating | NF |
| Cellulose | | | |
| Methacrylic acid | Eudragit RS PO | Film Coating | USP |
| copolymer | | | |
| Triethyl Citrate | Morflex | Plasticizer | USP |
| Amylose acetate | NA | Colonic Selective | In-House |
| | | Releasing Agent | |

### Example 8

Solid Oral Dosage Form

Solid oral dosage forms of 5-azacytidine were prepared using standard pharmaceutical excipients and techniques. TPGS was first adsorbed onto either microcrystalline cellulose or calcium silicate in an independent step. Dry ingredients were then dry blended and tablets prepared by direct compression. Tablets were then film coated with Klucel EF from ethanol followed by film coating with a polymer of 2-hydroxyethyl methacrylic acid cross linked with divinyl azobenzene (HEMA-DVAB polymer) and triethyl citrate from an ethanol—diethyl ether solvent mixture.

Clinical Trial Material cores were composed of the following materials in these ratios:

| Ingredient | mg/tablet | % w/w |
|---|---|---|
| 5-azacytidine | 20.0 | 20.0 |
| Mannitol, USP | 43.2 | 43.2 |
| Microcrystalline Cellulose, NF | 30.0 | 30.0 |
| Crospovidone, NF | 3.0 | 3.0 |
| Magnesium Stearate, NF | 1.8 | 1.8 |
| Vitamin E TPGS, NF | 2.0 | 2.0 |

Cores were then sub-coated to approximately 4% w/w with the following materials in ethanol:

| Ingredient | mg/tablet | % w/w |
|---|---|---|
| Klucel EF, NF | 4.0 | 6.0 |

Film coated cores were then be coated to approximately 6% w/w with the following mixture:

| Ingredient | mg/tablet | % w/w |
|---|---|---|
| HEMA-DVAB Polymer | 5.0 | 83.3 |
| Triethyl citrate, NF | 1.0 | 16.7 |

Excipient compatibility studies have demonstrated that 5-azacytidine is compatible with each excipient. Stability studies have demonstrated excellent stability of coated tablets under long term (25° C., 60% RH) and accelerated (40°, 70% RH) storage conditions. An additional formulation composition that has been manufactured is presented in Table 9.

TABLE 9
Oral 5-azacytidine Tablet Composition

| | | | Quality |
|---|---|---|---|
| Material | Trade Name | Purpose | Standard |
| 5-azacytidine | NA | Active | In-House |
| Mannitol | Parteck M200 | Bulking Agent | USP |
| Microcrystalline | Prosolv 90HD | Binding Agent | USP |
| Cellulose | | | |
| Crospovidone | Polyplasone XL | Disintegrant | USP |
| Magnesium Stearate | NA | Lubricant | USP |
| Vitamin E TPGS | NA | Absorption | NF |
| | | Enhancement | |
| Hydroxypropyl | Klucel EF | Sub-Coating | NF |
| Cellulose | | | |
| HEMA-DVAB | NA | Colonic Selective | In-House |
| polymer | | Releasing Agent | |
| Triethyl Citrate | Morflex | Plasticizer | USP |

### Example 9

Solid Oral Dosage Form

Solid oral dosage forms of 5-azacytidine were prepared using standard pharmaceutical excipients and techniques. TPGS was first adsorbed onto either microcrystalline cellulose or calcium silicate in an independent step. Dry ingredients were then dry blended for subsequent encapsulation into water impermeable, crosslinked gelatin capsules. The open capsule end was then sealed with Eudragit RL PO, a water insoluble, pH independent, swelling polymethacrylate polymer and triethyl citrate from ethanol. The amount of Eudragit RL PO polymer used to seal the capsule end was sufficient to require 3 hours or exposure to water prior to release of the capsule contents.

Clinical Trial Material capsules were composed of the following materials in these ratios:

| Ingredient | mg/capsule | % w/w |
|---|---|---|
| 5-azacytidine | 20.0 | 20.0 |
| Mannitol, USP | 33.2 | 33.2 |
| Microcrystalline Cellulose, NF | 40.0 | 40.0 |
| Crospovidone, NF | 3.0 | 3.0 |
| Magnesium Stearate, NF | 1.8 | 1.8 |
| Vitamin E TPGS, NF | 2.0 | 2.0 |

Filled capsules were then sealed with approximately 11.5% w/w the following mixture of Eudragit RL PO/triethyl citrate:

| Ingredient | mg/capsule | % w/w |
|---|---|---|
| Eudragit RL PO, NF | 10.0 | 87.0 |
| Triethyl citrate, NF | 1.5 | 13.0 |

Excipient compatibility studies have demonstrated that 5-azacytidine is compatible with each excipient. Stability studies have demonstrated excellent stability of coated tablets under long term (25° C., 60% RH) and accelerated (40°, 70% RH) storage conditions. An additional formulation composition that has been manufactured is presented in Table 10.

TABLE 10
Oral 5-azacytidine Tablet Composition

| | | | Quality |
|---|---|---|---|
| Material | Trade Name | Purpose | Standard |
| 5-azacytidine | NA | Active | In-House |
| Mannitol | Parteck M200 | Bulking Agent | USP |
| Microcrystalline | Prosolv 90HD | Binding Agent | USP |
| Cellulose | | | |
| Crospovidone | Polyplasone XL | Disintegrant | USP |
| Magnesium Stearate | NA | Lubricant | USP |
| Vitamin E TPGS | NA | Absorption | NF |
| | | Enhancement | |
| Hydroxypropyl | Klucel EF | Sub-Coating | NF |
| Cellulose | | | |
| Methacrylic acid | Eudragit RL PO | Film Coating | USP |
| copolymer¹ | | | |
| Triethyl Citrate | Morflex | Plasticizer | USP |

### Example 10

A clinical study using each of the oral formulations described in Examples 6-9 is performed to assess the safety and bioavailability of single oral doses of 5-azacytidine in patients with myelodysplastic syndromes, acute myelogenous leukemia, or solid tumors. The study is a multicenter, open-label, single treatment study. One patient receives an oral dose of the study drug starting at 60 mg. Subsequent patients are treated with escalating doses, in 20 mg increments, up to 200 mg. The study assesses the safety and tolerability of escalating doses, provides pilot information on the oral bioavailability of the study drug, and provides information on the single dose pharmacokinetics of the study drug after oral administration.

The outcome from the clinical trial is shown to be that patients dosed with the current oral 5-azacytidine formulation show measurable levels of 5-azacytidine in plasma samples. The amount of 5-azacytidine measured in the plasma is proportional to the administered dose, and the apparent oral bioavailability is acceptable for therapeutic treatment. The conclusions drawn from such results are that the current formulation is delivering 5-azacytidine to the colonic region, and that site is capable of efficiently absorbing 5-azacytidine without the degradation associated with cytidine deaminase. Ultimately, such observations lead to broader clinical applications of 5-azacytidine.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A controlled release pharmaceutical composition for oral administration of a cytidine analog comprising a) a therapeutically effective amount of a cytidine analog and b) a drug release controlling component capable of providing release of the cytidine analog primarily in the large intestine, wherein after ingestion by a patient the cytidine analog is released primarily in the large intestine.

2. The pharmaceutical composition of claim 1, wherein at least about 70% of the cytidine analog is released in the large intestine.

3. The pharmaceutical composition of claim 1, wherein the drug release controlling component is selected from the group consisting of an enteric component, a time delay component, a bacterially degradable component, and mixtures thereof.

4. The pharmaceutical composition of claim 3, wherein the drug release controlling component is an enteric coating, and wherein the enteric coating does not substantially dissolve in aqueous solution at a pH of above about pH 6.4 for at least about two hours.

5. The pharmaceutical composition of claim 3, wherein the enteric coating material comprises an agent selected from the group consisting of any grade of hydroxypropylmethylcellulose phthalate, polyvinyl acetate phthalate (PVAP), hydroxypropylmethylcellulose acetate succinate (HPMCAS), alginate, carbomer, carboxymethyl cellulose, methacrylic acid copolymer, shellac, cellulose acetate phthalate (CAP), starch glycolate, polacrylin, methyl cellulose acetate phthalate, hydroxymethylcellulose phthalate, hydroxymethylmethylcellulose acetate succinate, hydroxypropylcellulose acetate phthalate, cellulose acetate terephthalate, cellulose acetate isophthalate, cellulose acetate trimellitate, and mixtures thereof.

6. The pharmaceutical composition of claim 5, wherein the methacrylic acid copolymer is selected from the group consisting of a cationic copolymer of dimethyl aminoethyl methacrylate and neutral methacrylic esters, trimethylaminoethylmethacrylate and neutral methacrylic esters, and anionic polymers of methacrylic acid and methacrylates with carboxyl functional groups.

7. The pharmaceutical composition of claim 4, comprising
(a) a drug core and seal coat, wherein the drug core comprises 5-azacytidine, in an amount of at least about 20% w/w of the drug core and seal coat, further comprising at least one of the following excipients: diluent, binding agent, lubricant, disintegrant, and stabilizer and further comprising a seal coat, in an amount sufficient to form a sealed drug core; and
(b) an enteric coat, in an additional amount of between about 2% and 20% w/w relative to the drug core and seal coat, wherein the enteric coat comprises an anionic polymer of methacrylic acid and methacrylates with carboxyl functional groups with a threshold pH of about 6.8 (EUDRAGIT S100), in an amount of between about 60% and about 95% w/w of the enteric coat, and optionally further comprising a plasticizer, in an amount of between about 5% and about 40% w/w of the enteric coat.

8. The pharmaceutical composition of claim 7, wherein the excipients comprise at least one of the following: (a) a diluent, wherein the diluent comprises mannitol, in an amount of about 43% w/w of the drug core and seal coat; (b) a binding agent, wherein the binding agent comprises microcrystalline cellulose, in an amount of about 30% w/w of the drug core and seal coat; (c) a disintegrant, wherein the disintegrant comprises crospovidone, in an amount of about 3% w/w of the drug core and seal coat; (d) a lubricant, wherein the lubricant comprises magnesium stearate, in an amount of about 1.8% w/w of the drug core and seal coat, (e) a stabilizer, wherein the stabilizer comprises Vitamin E TPGS, in an amount of about 2% w/w of the drug core and seal coat; wherein the seal coat comprises hydroxypropyl cellulose, in an amount of about 6% w/w of the drug core and seal coat; and wherein the enteric coat comprises an additional amount of about 7% w/w relative to the drug core and seal coat, and the anionic polymer of methacrylic acid and methacrylates with carboxyl functional groups with a threshold pH of about 6.8, in an amount of about 86% w/w of the enteric coat and wherein the enteric coat further comprises a plasticizer comprising triethyl citrate, in an amount of about 14% w/w of the enteric coat.

9. The controlled release pharmaceutical composition of claim 3, wherein the drug release controlling component comprises a time delay component, and wherein the time delay component does not allow substantial release of the cytidine analog for at least about three hours after oral ingestion by a patient.

10. The controlled release pharmaceutical composition of claim 9, wherein the time delay component is a matrix or coating and is selected from the group consisting a poorly soluble polymer selected from the group consisting of polyvinyl chloride, polyethylene, vinyl polymers and copolymers selected from the group consisting of polyvinyl pyrrolidone, polyvinyl acetate, polyvinylacetate phthalate, vinylacetate crotonic acid copolymer, and ethylene-vinyl acetate copolymer; hydroxypropyl methyl cellulose, shellac, ammoniated shellac, shellac-acetyl alcohol, shellac n-butyl stearate, and copolymers of acrylic and methacrylic acid esters with a low content in quaternary ammonium groups with an average molecular weight of about 150,000 D (EUDRAGIT RS PO).

11. The pharmaceutical composition of claim 9, comprising
(a) a drug core and seal coat, wherein the drug core comprises 5-azacytidine, in an amount of at least about 20% w/w of the drug core and seal coat, further comprising at least one of the following excipients: diluent, binding agent, lubricant, disintegrant, stabilizer; and further comprising a seal coat, in an amount sufficient to form a sealed drug core; and
(b) a time delay coat, in an additional amount of between about 2% and about 20% w/w relative to the drug core and seal coat, wherein the time delay coat comprises copolymers of acrylic and methacrylic acid esters with a low content in quaternary ammonium groups with an average molecular weight of about 150,000 D (EUDRAGIT RS PO), in an amount of between about 60% and about 95% w/w of the time delay coat, and optionally further comprising a plasticizer, in an amount of between about 5% and 40% w/w of the time delay coat.

12. The pharmaceutical composition of claim 11, wherein the excipients comprise at least one of the following: (a) a diluent, wherein the diluent comprises mannitol, in an amount of about 33% w/w of the drug core and seal coat; (b) a binding agent, wherein the binding agent comprises microcrystalline cellulose, in an amount of about 40% w/w of the drug core and seal coat; (c) a disintegrant, wherein the disintegrant comprises crospovidone, in an amount of about 3% w/w of the drug core and seal coat; (d) a lubricant, wherein the lubricant comprises magnesium stearate, in an amount of about 1.8% w/w of the drug core and seal coat, (e) a stabilizer, wherein the stabilizer comprises Vitamin E TPGS, in an amount of about 2% w/w of the drug core and seal coat; wherein the seal coat comprises hydroxypropyl cellulose, in an amount of about 5% w/w of the drug core and seal coat; and wherein the time coat comprises an additional amount of about 11.5% w/w relative to the drug core and seal coat, and wherein the copolymers of acrylic and methacrylic acid esters with a low content in quaternary ammonium groups with an average molecular weight of about 150,000 D (EUDRAGIT RS PO) are in an amount of about 87% w/w of the time delay coat and wherein the coat further comprises a plasticizer, wherein the plasticizer is triethyl citrate, in an amount of about 13% w/w of the time delay coat.

13. The controlled release pharmaceutical composition of claim 3, wherein the drug release controlling component comprises a bacterially degradable component, wherein patients lack the digestive enzymes required to degrade the component.

14. The controlled release pharmaceutical component of claim 13, wherein the bacterially degradable component is selected from the group consisting of a polymer of 2-hydroxyethyl methacrylic acid cross linked with divinyl azobenzene (HEMA-DVAB polymer), chitosan, amylose, cellobiose, lactulose, raffinose and stachyose, and polymers thereof.

15. The pharmaceutical composition of claim 9, comprising
(a) a drug core and seal coat, wherein the drug core comprises 5-azacytidine, in an amount of at least about 20% w/w of the drug core and seal coat, and further comprising at least one of the following excipients: diluent, binding agent, lubricant, disintegrant, stabilizer, and further comprising a seal coat, in an amount sufficient to form a sealed drug core; and
(b) a bacterially degradable coat, in an additional amount of between about 2% and 20% w/w relative to the drug core and seal coat, wherein the bacterially degradable coat comprises one of the following formulations:
(i) copolymers of acrylic and methacrylic acid esters with a low content in quaternary ammonium groups with an average molecular weight of about 150,000 D (EUDRAGIT RS PO), in an amount of between about 30% and about 70% w/w of the bacterially degradable coat; optionally further comprising a plasticizer, in an amount of between about 5% and 40% w/w of the bacterially degradable coat; pectin, in an amount of between about 10% and about 30% w/w of the bacterially degradable coat, and chitosan, in an amount of between about 10% and about 30% w/w of the bacterially degradable coat; and
(ii) copolymers of acrylic and methacrylic acid esters with a low content in quaternary ammonium groups with an average molecular weight of about 150,000 D (EUDRAGIT RS PO), in an amount of between about 30% and about 70% w/w of the bacterially degradable coat; optionally further comprising a plasticizer, in an amount of between about 5% and 40% w/w of the bacterially degradable coat; and amylose, in an amount of between about 30% and about 60% w/w of the bacterially degradable coat.

16. The pharmaceutical composition of claim 15, wherein the excipients comprise at least one of the following (a) diluent, wherein the diluent comprises mannitol, in an amount of about 43% w/w of the drug core and seal coat; (b) a binding agent, wherein the binding agent comprises microcrystalline cellulose, in an amount of about 30% w/w of the drug core and seal coat; (c) disintegrant, wherein the disintegrant comprises crospovidone, in an amount of about 3% w/w of the drug core and seal coat; (d) lubricant, wherein the lubricant comprises magnesium stearate, in an amount of about 1.8% w/w of the drug core and seal coat, (e) stabilizer, wherein the stabilizer comprises Vitamin E TPGS, in an amount of about 2% w/w of the drug core and seal coat; further comprising a seal coat, wherein the seal coat comprises hydroxypropyl cellulose, in an amount of about 4% w/w of the drug core and seal coat; and wherein the bacterially degradable coat in (i) comprises an additional amount of about 9% w/w relative to the drug core and seal coat of a mixture of copolymers of acrylic and methacrylic acid esters with a low content in quaternary ammonium groups with an average molecular weight of about 150,000 D (EUDRAGIT RS PO) in an amount of about 56% w/w of the bacterially degradable coat, a plasticizer, wherein the plasticizer is triethyl citrate in an amount of about 11% w/w of the bacterially degradable coat, pectin, in an amount of about 17% w/w of the bacterially degradable coat; and chitosan, in an amount of about 17% w/w of the bacterially degradable coat; and wherein the bacterially degradable coat in (ii) comprises an additional amount of about 9% w/w relative to the drug core and seal coat of a mixture of copolymers of acrylic and methacrylic acid esters with a low content in quaternary ammonium groups with an average molecular weight of about 150,000 D (EUDRAGIT RS PO) in an amount of about 45% w/w of the bacterially degradable coat, a plasticizer, wherein the plasticizer is triethyl citrate in an amount of about 8% w/w of the bacterially degradable coat, and amylose, in an amount of about 47% w/w of the bacterially degradable coat.

17. The pharmaceutical composition of claim 9, comprising
(a) a drug core and seal coat, wherein the drug core comprises 5-azacytidine, in an amount of least about 20% w/w of the drug core and seal coat, further comprising at least one of the following excipients: diluent, binding agent, lubricant, disintegrant, stabilizer and further comprising a seal coat, in an amount sufficient to form a sealed drug core; and
(c) a bacterially degradable coat, in an additional amount of between about 2% and about 20% w/w relative to the drug core and seal coat, wherein the bacterially degradable coat comprises a polymer of 2-hydroxyethyl methacrylic acid cross linked with divinyl azobenzene (HEMA-DVAB polymer), in an amount of between about 60% and about 95% w/w of the bacterially degradable coat, and optionally further comprising a plasticizer, in an amount of between about 5% and about 40% w/w of the bacterially degradable coat.

18. The pharmaceutical composition of claim 17, wherein the excipients comprise at least one of the following: (a) a diluent, wherein the diluent comprises mannitol, in an amount of about 43% w/w of the drug core and seal coat, (b) a binding agent, wherein the binding agent comprises microcrystalline cellulose, in an amount of about 30% w/w of the drug core and seal coat; (c) disintegrant, wherein the disintegrant comprises crospovidone, in an amount of about 3% w/w of the drug core and seal coat, (d) lubricant, wherein the lubricant comprises magnesium stearate, in an amount of about 1.8% w/w of the drug core and seal coat, (e) stabilizer, wherein the stabilizer comprises Vitamin E TPGS, in an amount of about 2% w/w of the drug core and seal coat; wherein the seal coat comprises hydroxypropyl cellulose, in an amount of about 4% w/w of the drug core and seal coat; and wherein the bacterially degradable coat is in an additional amount about 6% w/w relative to the drug core and seal coat, and the polymer of 2-hydroxyethyl methacrylic acid cross linked with divinyl azobenzene (HEMA-DVAB polymer) in an amount of about 83% w/w of the bacterially degradable coat, and wherein the bacterially degradable coat further comprises a plasticizer, wherein the plasticizer is triethyl citrate in an amount of about 17% w/w of the bacterially degradable coat.

19. The pharmaceutical composition of claim 1, wherein the cytidine analog is selected from the group consisting of 5-aza-2′-deoxycytidine (decitabine), 5-azacytidine, 5-aza-2′-deoxy-2′,2′-difluorocytidine, 5-aza-2′-deoxy-2′-fluorocytidine, 2′-deoxy-2′,2′-difluorocytidine (also called gemcitabine), or cytosine 1-β-D-arabinofuranoside (also called ara-C), 2(1H) pyrimidine riboside (also called zebularine), 2′-cyclocytidine, arabinofuanosyl-5-azacytidine, dihydro-5-azacytidine, N⁴-octadecyl-cytarabine, and elaidic acid cytarabine.

20. The pharmaceutical composition of claim 19, wherein the cytidine analog is 5-azacytidine.

21. A method for treating a patient having a disease associated with abnormal cell proliferation, comprising: orally administering to the patient a pharmaceutical composition in accordance with claim 1.

22. The method of claim 21, wherein the disease associated with abnormal cell proliferation is a myelodysplastic syndrome.

23. A method for delivering a cytidine analog comprising administering to a patient in need thereof an oral formulation of a cytidine analog, wherein the oral formulation of the cytidine analog comprises a) a therapeutically effective amount of a cytidine analog and b) a drug release controlling component capable of providing release of the cytidine analog primarily in the large intestine, wherein after ingestion by a patient the cytidine analog is released primarily in the large intestine.

24. The method of claim 23, wherein the drug release controlling component is selected from the group consisting of an enteric component, a time delay component, a bacterially degradable component, and mixtures thereof.

25. The method of claim 23, wherein the drug release controlling component is an enteric coating, and wherein the enteric coating does not substantially dissolve in aqueous solution at a pH of above about pH 6.4 for at least about two hours.

26. The method of claim 23, wherein the drug release controlling component comprises a time delay component, and wherein the time delay component does not allow substantial release of the cytidine analog for at least about three hours after oral ingestion by a patient.

27. The method of claim 23, wherein the drug release controlling component comprises a bacterially degradable component, wherein patients lack the digestive enzymes required to degrade the component.

28. The method of claim 23, wherein the cytidine analog is 5-azacytidine.

29. The method of claim 23, wherein the patient has a myelodysplastic syndrome.

30. A method of formulating a cytidine analog for oral delivery, comprising coating a therapeutically effective amount of a cytidine analog with a drug release controlling component capable of providing release of the cytidine analog primarily in the large intestine.

31. The method of claim 30, wherein the drug release controlling component is selected from the group consisting of an enteric component, a time delay component, a bacterially degradable component, and mixtures thereof.

32. The method of claim 30, wherein the drug release controlling component is an enteric coating, and wherein the enteric coating does not substantially dissolve in aqueous solution at a pH of above about pH 6.4 for at least about two hours.

33. The method of claim 30, wherein the drug release controlling component comprises a time delay component, and wherein the time delay component does not allow substantial release of the cytidine analog for at least about three hours after oral ingestion by a patient.

34. The method of claim 30, wherein the drug release controlling component comprises a bacterially degradable component, wherein patients lack the digestive enzymes required to degrade the component.

35. The method of claim 30, wherein the cytidine analog is 5-azacytidine.

36. A method of increasing the bioavailability of a cytidine analog upon administration to a patient, comprising: (I) providing a controlled release pharmaceutical composition to a patient, comprising a) a therapeutically effective amount of a cytidine analog and b) a drug release controlling component capable of providing release of the cytidine analog primarily in the large intestine, wherein after ingestion by a patient the cytidine analog is released primarily in the large intestine; and (II) ingesting of said composition by the patient, whereby said composition contacts the biological fluids of the patient's body and increases the bioavailability of the cytidine analog.

37. The method of claim 36, wherein the drug release controlling component is selected from the group consisting of an enteric component, a time delay component, a bacterially degradable component, and mixtures thereof.

38. The method of claim 36, wherein the drug release controlling component is an enteric coating, and wherein the enteric coating does not substantially dissolve in aqueous solution at a pH of above about pH 6.4 for at least about two hours.

39. The method of claim 36, wherein the drug release controlling component comprises a time delay component, and wherein the time delay component does not allow substantial release of the cytidine analog for at least about three hours after oral ingestion by a patient.

40. The method of claim 36, wherein the drug release controlling component comprises a bacterially degradable component, wherein patients lack the digestive enzymes required to degrade the component.

41. The method of claim 36, wherein the cytidine analog is 5-azacytidine.

42. The method of claim 36, wherein the patient has a myelodysplastic syndrome.
